SEQUENCE LISTING (64 sequences)

SEQ ID NO: 1 - DNA, 82 bp, Artificial Sequence
  Synthetic Construct
  misc_feature (22)..(61): n is a, c, g, or t
caggtccatcgagtggtaggannnnnnnnnnnnnnnnnnnnnnnnnnnnnnnnnnnnnnnntcgcactgctcctgaacgtac

SEQ ID NO: 2 - DNA, 21 bp, Artificial Sequence
  Synthetic Construct
caggtccatcgagtggtagga

SEQ ID NO: 3 - DNA, 21 bp, Artificial Sequence
  Synthetic Construct
gtacgttcaggagcagtgcga

SEQ ID NO: 4 - DNA, 34 bp, Artificial Sequence
  Synthetic Construct
  misc_feature (13)..(13): n = phosphoramidite
aaaaaaaaaaaangtacgttcaggagcagtgcga

SEQ ID NO: 5 - DNA, 24 bp, Artificial sequence
  Synthetic Construct
tcgatggacctggtacgttcagga

SEQ ID NO: 6 - DNA, 82 bp, Artificial Sequence
  Synthetic Construct
caggtccatcgagtggtaggaggaggtatttagtgccaagccatctcaaacgacgtctgagtcgcactgctcctgaacgtac

SEQ ID NO: 7 - DNA, 82 bp, Artificial Sequence
  Synthetic Construct
caggtccatcgagtggtaggacgacccgaatagtggcatatcaatgagtgcttgtcatctttcgcactgctcctgaacgtac

SEQ ID NO: 8 - DNA, 82 bp, Artificial Sequence
  Synthetic Construct
caggtccatcgagtggtaggatgcagcggacagtgtgggaccatcgctgcggatgtatgaatcgcactgctcctgaacgtac

SEQ ID NO: 9 - DNA, 82 bp, Artificial Sequence
  Synthetic Construct
caggtccatcgagtggtaggacccaacccacgatgcgcaagaggaatgcagcctaccagcatcgcactgctcctgaacgtac

SEQ ID NO: 10 - DNA, 59 bp, Artificial Sequence
  Synthetic Construct
taggaggaggtatttagtgccaagccatctcaaacgacgtctgagtcgcactgctcctg

SEQ ID NO: 11 - DNA, 82 bp, Artificial Sequence
  Synthetic Construct
cgggagtacgcatgtatgagacacccacgctatgtccctactatgaggtggagacttgctacgtgcatcgcagatcggtcaa

SEQ ID NO: 12 - DNA, 82 bp, Artificial Sequence
  Synthetic Construct
gaaggattcggactaggatccggtccaactaccggtgccctaatggcatcaaaactgcgtaccgagcgtgtttatctttctg

SEQ ID NO: 13 - DNA, 40 bp, Artificial Sequence
  Synthetic Construct
cagctgtcgacgcgttaccgtgaacggaacaccgatgacg

SEQ ID NO: 14 - DNA, 40 bp, Artificial Sequence
  Synthetic Construct
ggaggtatttagtgccaagccatctcaaacgacgtctgag

SEQ ID NO: 15 - DNA, 40 bp, Artificial Sequence
  Synthetic Construct
gtgtagggacgcaagatgaatgcagcataccagtccctag

SEQ ID NO: 16 - DNA, 40 bp, Artificial Sequence
  Synthetic Construct
cgacccgaatagtggcatatcaatgagtgcttgtcatctt

SEQ ID NO: 17 - DNA, 40 bp, Artificial Sequence
  Synthetic Construct
tcagtaccagttccccaggagaatgcagatccccaggtac

SEQ ID NO: 18 - protein, 421 aa, Clostridium difficile
MSGKDVNVFEMAQSQVKNACDKLGMEPAVYELLKEPMRVIEVSIPVKMDDGSIKTFKGFRSQHNDAVGPTKGGIRFHQNVSRDEVKALSIWMTFKCSVTGIPYGGGKGGIIVDPSTLSQGELERLSRGYIDGIYKLIGEKVDVPAPDVNTNGQIMSWMVDEYNKLTGQSSIGVITGKPVEFGGSLGRTAATGFGVAVTAREAAAKLGIDMKKAKIAVQGIGNVGSYTVLNCEKLGGTVVAMAEWCKSEGSYAIYNENGLDGQAMLDYMKEHGNLLNFPGAKRISLEEFWASDVDIVIPAALENSITKEVAESIKAKLVCEAANGPTTPEADEVFAERGIVLTPDILTNAGGVTVSYFEWVQNLYGYYWSEEEVEQKEEIAMVKAFESIWKIKEEYNVTMREAAYMHSIKKVAEAMKLRGWY

SEQ ID NO: 19 - protein, 2710 aa, Clostridium difficile
MSLISKEELIKLAYSIRPRENEYKTILTNLDEYNKLTTNNNENKYLQLKKLNESIDVFMNKYKTSSRNRALSNLKKDILKEVILIKNSNTSPVEKNLHFVWIGGEVSDIALEYIKQWADINAEYNIKLWYDSEAFLVNTLKKAIVESSTTEALQLLEEEIQNPQFDNMKFYKKRMEFIYDRQKRFINYYKSQINKPTVPTIDDIIKSHLVSEYNRDETVLESYRTNSLRKINSNHGIDIRANSLFTEQELLNIYSQELLNRGNLAAASDIVRLLALKNFGGVYLDVDMLPGIHSDLFKTISRPSSIGLDRWEMIKLEAIMKYKKYINNYTSENFDKLDQQLKDNFKLIIESKSEKSEIFSKLENLNVSDLEIKIAFALGSVINQALISKQGSYLTNLVIEQVKNRYQFLNQHLNPAIESDNNFTDTTKIFHDSLFNSATAENSMFLTKIAPYLQVGFMPEARSTISLSGPGAYASAYYDFINLQENTIEKTLKASDLIEFKFPENNLSQLTEQEINSLWSFDQASAKYQFEKYVRDYTGGSLSEDNGVDFNKNTALDKNYLLNNKIPSNNVEEAGSKNYVHYIIQLQGDDISYEATCNLFSKNPKNSIIIQRNMNESAKSYFLSDDGESILELNKYRIPERLKNKEKVKVTFIGHGKDEFNTSEFARLSVDSLSNEISSFLDTIKLDISPKNVEVNLLGCNMFSYDFNVEETYPGKLLLSIMDKITSTLPDVNKNSITIGANQYEVRINSEGRKELLAHSGKWINKEEAIMSDLSSKEYIFFDSIDNKLKAKSKNIPGLASISEDIKTLLLDASVSPDTKFILNNLKLNIESSIGDYIYYEKLEPVKNIIHNSIDDLIDEFNLLENVSDELYELKKLNNLDEKYLISFEDISKNNSTYSVRFINKSNGESVYVETEKEIFSKYSEHITKEISTIKNSIITDVNGNLLDNIQLDHTSQVNTLNAAFFIQSLIDYSSNKDVLNDLSTSVKVQLYAQLFSTGLNTIYDSIQLVNLISNAVNDTINVLPTITEGIPIVSTILDGINLGAAIKELLDEHDPLLKKELEAKVGVLAINMSLSIAATVASIVGIGAEVTIFLLPIAGISAGIPSLVNNELILHDKATSVVNYFNHLSESKKYGPLKTEDDKILVPIDDLVISEIDFNNNSIKLGTCNILAMEGGSGHTVTGNIDHFFSSPSISSHIPSLSIYSAIGIETENLDFSKKIMMLPNAPSRVFWWETGAVPGLRSLENDGTRLLDSIRDLYPGKFYWRFYAFFDYAITTLKPVYEDTNIKIKLDKDTRNFIMPTITTNEIRNKLSYSFDGAGGTYSLLLSSYPISTNINLSKDDLWIFNIDNEVREISIENGTIKKGKLIKDVLSKIDINKNKLIIGNQTIDFSGDIDNKDRYIFLTCELDDKISLIIEINLVAKSYSLLLSGDKNYLISNLSNTIEKINTLGLDSKNIAYNYTDESNNKYFGAISKTSQKSIIHYKKDSKNILEFYNDSTLEFNSKDFIAEDINVFMKDDINTITGKYYVDNNTDKSIDFSISLVSKNQVKVNGLYLNESVYSSYLDFVKNSDGHHNTSNFMNLFLDNISFWKLFGFENINFVIDKYFTLVGKTNLGYVEFICDNNKNIDIYFGEWKTSSSKSTIFSGNGRNVVVEPIYNPDTGEDISTSLDFSYEPLYGIDRYINKVLIAPDLYTSLININTNYYSNEYYPEIIVLNPNTFHKKVNINLDSSSFEYKWSTEGSDFILVRYLEESNKKILQKIRIKGILSNTQSFNKMSIDFKDIKKLSLGYIMSNFKSFNSENELDRDHLGFKIIDNKTYYYDEDSKLVKGLININNSLFYFDPIEFNLVTGWQTINGKKYYFDINTGAALTSYKIINGKHFYFNNDGVMQLGVFKGPDGFEYFAPANTQNNNIEGQAIVYQSKFLTLNGKKYYFDNNSKAVTGWRIINNEKYYFNPNNAIAAVGLQVIDNNKYYFNPDTAIISKGWQTVNGSRYYFDTDTAIAFNGYKTIDGKHFYFDSDCVVKIGVFSTSNGFEYFAPANTYNNNIEGQAIVYQSKFLTLNGKKYYFDNNSKAVTGLQTIDSKKYYFNTNTAEAATGWQTIDGKKYYFNTNTAEAATGWQTIDGKKYYFNTNTAIASTGYTIINGKHFYFNTDGIMQIGVFKGPNGFEYFAPANTDANNIEGQAILYQNEFLTLNGKKYYFGSDSKAVTGWRIINNKKYYFNPNNAIAAIHLCTINNDKYYFSYDGILQNGYITIERNNFYFDANNESKMVTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTDANNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAPGIYG

SEQ ID NO: 20 - protein, 2366 aa, Clostridium difficile
MSLVNRKQLEKMANVRFRTQEDEYVAILDALEEYHNMSENTVVEKYLKLKDINSLTDIYIDTYKKSGRNKALKKFKEYLVTEVLELKNNNLTPVEKNLHFVWIGGQINDTAINYINQWKDVNSDYNVNVFYDSNAFLINTLKKTVVESAINDTLESFRENLNDPRFDYNKFFRKRMEIIYDKQKNFINYYKAQREENPELIIDDIVKTYLSNEYSKEIDELNTYIEESLNKITQNSGNDVRNFEEFKNGESFNLYEQELVERWNLAAASDILRISALKEIGGMYLDVDMLPGIQPDLFESIEKPSSVTVDFWEMTKLEAIMKYKEYIPEYTSEHFDMLDEEVQSSFESVLASKSDKSEIFSSLGDMEASPLEVKIAFNSKGIINQGLISVKDSYCSNLIVKQIENRYKILNNSLNPAISEDNDFNTTTNTFIDSIMAEANADNGRFMMELGKYLRVGFFPDVKTTINLSGPEAYAAAYQDLLMFKEGSMNIHLIEADLRNFEISKTNISQSTEQEMASLWSFDDARAKAQFEEYKRNYFEGSLGEDDNLDFSQNIVVDKEYLLEKISSLARSSERGYIHYIVQLQGDKISYEAACNLFAKTPYDSVLFQKNIEDSEIAYYYNPGDGEIQEIDKYKIPSIISDRPKIKLTFIGHGKDEFNTDIFAGFDVDSLSTEIEAAIDLAKEDISPKSIEINLLGCNMFSYSINVEETYPGKLLLKVKDKISELMPSISQDSIIVSANQYEVRINSEGRRELLDHSGEWINKEESIIKDISSKEYISFNPKENKITVKSKNLPELSTLLQEIRNNSNSSDIELEEKVMLTECEINVISNIDTQIVEERIEEAKNLTSDSINYIKDEFKLIESISDALCDLKQQNELEDSHFISFEDISETDEGFSIRFINKETGESIFVETEKTIFSEYANHITEEISKIKGTIFDTVNGKLVKKVNLDTTHEVNTLNAAFFIQSLIEYNSSKESLSNLSVAMKVQVYAQLFSTGLNTITDAAKVVELVSTALDETIDLLPTLSEGLPIIATIIDGVSLGAAIKELSETSDPLLRQEIEAKIGIMAVNLTTATTAIITSSLGIASGFSILLVPLAGISAGIPSLVNNELVLRDKATKVVDYFKHVSLVETEGVFTLLDDKIMMPQDDLVISEIDFNNNSIVLGKCEIWRMEGGSGHTVTDDIDHFFSAPSITYREPHLSIYDVLEVQKEELDLSKDLMVLPNAPNRVFAWETGWTPGLRSLENDGTKLLDRIRDNYEGEFYWRYFAFIADALITTLKPRYEDTNIRINLDSNTRSFIVPIITTEYIREKLSYSFYGSGGTYALSLSQYNMGINIELSESDVWIIDVDNVVRDVTIESDKIKKGDLIEGILSTLSIEENKIILNSHEINFSGEVNGSNGFVSLTFSILEGINAIIEVDLLSKSYKLLISGELKILMLNSNHIQQKIDYIGFNSELQKNIPYSFVDSEGKENGFINGSTKEGLFVSELPDVVLISKVYMDDSKPSFGYYSNNLKDVKVITKDNVNILTGYYLKDDIKISLSLTLQDEKTIKLNSVHLDESGVAEILKFMNRKGNTNTSDSLMSFLESMNIKSIFVNFLQSNIKFILDANFIISGTTSIGQFEFICDENDNIQPYFIKFNTLETNYTLYVGNRQNMIVEPNYDLDDSGDISSTVINFSQKYLYGIDSCVNKVVISPNIYTDEINITPVYETNNTYPEVIVLDANYINEKINVNINDLSIRYVWSNDGNDFILMSTSEENKVSQVKIRFVNVFKDKTLANKLSFNFSDKQDVPVSEIILSFTPSYYEDGLIGYDLGLVSLYNEKFYINNFGMMVSGLIYINDSLYYFKPPVNNLITGFVTVGDDKYYFNPINGGAASIGETIIDDKNYYFNQSGVLQTGVFSTEDGFKYFAPANTLDENLEGEAIDFTGKLIIDENIYYFDDNYRGAVEWKELDGEMHYFSPETGKAFKGLNQIGDYKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISE

SEQ ID NO: 21 - DNA, 21 bp, Artificial Sequence
  Synthetic Construct
caggtccatcgagtggtagga

SEQ ID NO: 22 - DNA, 20 bp, Artificial Sequence
  Synthetic Construct
aggtccatcgagtggtagga

SEQ ID NO: 23 - DNA, 19 bp, Artificial Sequence
  Synthetic Construct
ggtccatcgagtggtagga

SEQ ID NO: 24 - DNA, 18 bp, Artificial Sequence
  Synthetic Construct
gtccatcgagtggtagga

SEQ ID NO: 25 - DNA, 17 bp, Artificial Sequence
  Synthetic Construct
tccatcgagtggtagga

SEQ ID NO: 26 - DNA, 16 bp, Artificial Sequence
  Synthetic Construct
ccatcgagtggtagga

SEQ ID NO: 27 - DNA, 15 bp, Artificial Sequence
  Synthetic Construct
catcgagtggtagga

SEQ ID NO: 28 - DNA, 14 bp, Artificial Sequence
  Synthetic Construct
atcgagtggtagga

SEQ ID NO: 29 - DNA, 13 bp, Artificial Sequence
  Synthetic Construct
tcgagtggtagga

SEQ ID NO: 30 - DNA, 12 bp, Artificial Sequence
  Synthetic Construct
cgagtggtagga

SEQ ID NO: 31 - DNA, 11 bp, Artificial Sequence
  Synthetic Construct
gagtggtagga

SEQ ID NO: 32 - DNA, 10 bp, Artificial Sequence
  Synthetic Construct
agtggtagga

SEQ ID NO: 33 - DNA, 9 bp, Artificial Sequence
  Synthetic Construct
gtggtagga

SEQ ID NO: 34 - DNA, 8 bp, Artificial Sequence
  Synthetic Construct
tggtagga

SEQ ID NO: 35 - DNA, 7 bp, Artificial Sequence
  Synthetic Construct
ggtagga

SEQ ID NO: 36 - DNA, 6 bp, Artificial Sequence
  Synthetic Construct
gtagga

SEQ ID NO: 37 - DNA, 5 bp, Artificial Sequence
  Synthetic Construct
tagga

SEQ ID NO: 38 - DNA, 4 bp, Artificial Sequence
  Synthetic Construct
agga

SEQ ID NO: 39 - DNA, 3 bp, Artificial Sequence
  Synthetic Construct
gga

SEQ ID NO: 40 - DNA, 2 bp, Artificial Sequence
  Synthetic Construct
ga

SEQ ID NO: 41 - DNA, 1 bp, Artificial Sequence
  Synthetic Construct
a

SEQ ID NO: 42 - DNA, 21 bp, Artificial Sequence
  Synthetic Construct
tcgcactgctcctgaacgtac

SEQ ID NO: 43 - DNA, 20 bp, Artificial Sequence
  Synthetic Construct
tcgcactgctcctgaacgta

SEQ ID NO: 44 - DNA, 19 bp, Artificial Sequence
  Synthetic Construct
tcgcactgctcctgaacgt

SEQ ID NO: 45 - DNA, 18 bp, Artificial Sequence
  Synthetic Construct
tcgcactgctcctgaacg

SEQ ID NO: 46 - DNA, 17 bp, Artificial Sequence
  Synthetic Construct
tcgcactgctcctgaac

SEQ ID NO: 47 - DNA, 16 bp, Artificial Sequence
  Synthetic Construct
tcgcactgctcctgaa

SEQ ID NO: 48 - DNA, 15 bp, Artificial Sequence
  Synthetic Construct
tcgcactgctcctga

SEQ ID NO: 49 - DNA, 14 bp, Artificial Sequence
  Synthetic Construct
tcgcactgctcctg

SEQ ID NO: 50 - DNA, 13 bp, Artificial Sequence
  Synthetic Construct
tcgcactgctcct

SEQ ID NO: 51 - DNA, 12 bp, Artificial Sequence
  Synthetic Construct
tcgcactgctcc

SEQ ID NO: 52 - DNA, 11 bp, Artificial Sequence
  Synthetic Construct
tcgcactgctc

SEQ ID NO: 53 - DNA, 10 bp, Artificial Sequence
  Synthetic Construct
tcgcactgct

SEQ ID NO: 54 - DNA, 9 bp, Artificial Sequence
  Synthetic Construct
tcgcactgc

SEQ ID NO: 55 - DNA, 8 bp, Artificial Sequence
  Synthetic Construct
tcgcactg

SEQ ID NO: 56 - DNA, 7 bp, Artificial Sequence
  Synthetic Construct
tcgcact

SEQ ID NO: 57 - DNA, 6 bp, Artificial Sequence
  Synthetic Construct
tcgcac

SEQ ID NO: 58 - DNA, 5 bp, Artificial Sequence
  Synthetic Construct
tcgca

SEQ ID NO: 59 - DNA, 4 bp, Artificial Sequence
  Synthetic Construct
tcgc

SEQ ID NO: 60 - DNA, 3 bp, Artificial Sequence
  Synthetic Construct
tcg

SEQ ID NO: 61 - DNA, 2 bp, Artificial Sequence
  Synthetic Construct
tc

SEQ ID NO: 62 - DNA, 1 bp, Artificial Sequence
  Synthetic Construct
t

SEQ ID NO: 63 - DNA, 18 bp, Artificial Sequence
  Synthetic Construct
caggtccatcgagtggta

SEQ ID NO: 64 - DNA, 21 bp, Artificial Sequence
  Synthetic Construct
tcgcactgctcctgaacgtac

(19) United States
(12) Patent Application Publication
(10) Pub. No.: US 2020/0340042 A1
(43) Pub. Date: Oct. 29, 2020

(54) IN VITRO SELECTION FOR NUCLEIC ACID APTAMERS

(71) Applicant: McMaster University, Hamilton (CA)

(72) Inventors: Yingfu Li, Dundas (CA); John Brennan, Dundas (CA); Meng Liu, Dalian City (CN)

(21) Appl. No.: 16/857,885

(22) Filed: Apr. 24, 2020

Related U.S. Application Data
(60) Provisional application No. 62/837,837, filed on Apr. 24, 2019.

(51) Int. Cl. C12Q 1/689; C12Q 1/6844; C12N 15/113; C12Q 1/32; C12N 15/10

(52) U.S. Cl. CPC C12Q 1/689; C12Q 1/6844; C12N 15/113; C12Y 104/01002; C12N 15/1013; C12N 2310/16; C12Q 1/32

(57) ABSTRACT

Provided herein are methods for selection of circular aptamers using a circular nucleic acid library. Also provided are circular aptamers, circular aptamer probes, biosensor systems, and the methods for their use in detecting a microorganism target, or a target molecule present on or generated from a microorganism or a virus in a test sample, including C. difficile glutamate dehydrogenase and methods for determining whether a subject has a C. difficile infection.

### CROSS REFERENCE TO RELATED APPLICATIONS

The present disclosure claims priority from U.S. provisional application No. 62/837837 filed on Apr. 24, 2019, which is hereby incorporated by reference in its entirety.

### INCORPORATION OF SEQUENCE LISTING

A computer readable form of the Sequence Listing “3244-P58930US01_SequenceListing.txt” (60,940 bytes), submitted via EFS-WEB and created on Apr. 23, 2019, is herein incorporated by reference.

### FIELD

The present disclosure relates to the field of nucleic acid aptamers, and in particular, for methods of in vitro selection of circular aptamers and circular aptamers capable of binding to a target molecule, such as glutamate dehydrogenase produced by Clostridium difficile.

### BACKGROUND

Aptamers are single-stranded nucleic acids that contain sequence-dependent binding sites for defined molecular targets [1]. They are typically isolated from random-sequence libraries of nucleic acids via “Systematic Evolution of Ligands by Exponential Enrichment (SELEX)” [1,2]. One major advantage of SELEX is its adaptability with experimental conditions, as SELEX can be performed with diverse targets, assorted libraries and wide-ranging binding conditions. Aptamers also offer programmability based on predictable Watson-Crick base-pairing interactions and structure-switching capability, enabling them to be used as smart materials for bioanalytical and biomedical applications [3].

### SUMMARY

The present disclosure describes the selection of DNA aptamers from a circular DNA library. Use of a circular DNA library resulted in the discovery of two high-affinity circular DNA aptamers that recognize the glutamate dehydrogenase (GDH) from Clostridium difficile, an established antigen for diagnosing Clostridium difficile infection (CDI). One aptamer binds effectively in both the circular and linear forms, the other is functional only in the circular configuration. These two aptamers recognize different epitopes on GDH, demonstrating the advantage of selecting aptamers from circular DNA libraries. A sensitive diagnostic test was developed to take advantage of the high stability of circular DNA aptamers in biological samples and their compatibility with rolling circle amplification. This test was capable of identifying patients with active CDI using stool samples.

In accordance with a broad aspect of the present disclosure, there is provided a method comprising:

a) providing a plurality of circular nucleic acid molecules, wherein the plurality of circular nucleic acid molecules comprises oligonucleotides having at least one random nucleotide domain flanked by a 5′-end primer region and a 3′-end primer region;

b) contacting the plurality of circular nucleic acid molecules with a bare solid support;

c) collecting unbound circular nucleic acid molecules;

d) contacting the unbound circular nucleic acid molecules with solid support coated with the target molecule to form a complex comprising bound circular nucleic acid molecules;

e) optionally washing the complex;

f) eluting the bound circular nucleic acid molecules from the complex; and

g) amplifying the eluted circular nucleic acid molecules by polymerase chain reaction (PCR).

In an embodiment, the method further comprises repeating steps d) to g) for one to nineteen times, optionally for six to eleven times, with the amplified eluted circular nucleic acid molecules of g) as the unbound circular nucleic acid molecules in step d). In an embodiment, step a) providing a plurality of circular nucleic acid molecules comprises preparing a plurality of circular nucleic acid molecules by circularizing a plurality of linear nucleic acid molecules. In an embodiment, the solid support comprises nitrocellulose filter disc, optionally with a pore size 0.45 or 0.22 um, or magnetic beads coated with a metal, optionally Nickel Nitrilotriacetic acid magnetic beads (NiMBs). In an embodiment, step f) eluting comprises denaturing the circular nucleic acid molecules, optionally wherein the denaturing comprises heating or urea treatment, optionally 10M urea.

In an embodiment, the target molecule is a microorganism target, or a target molecule present on or generated from a microorganism or a virus. In an embodiment, the microorganism is selected from the group consisting of a bacteria, fungi, archaea, protists, algae, plankton and planarian. In an embodiment, the microorganism is a pathogenic bacterium. In an embodiment, the pathogenic bacterium is selected from the group consisting of Escherichia coli O157:H7, Listeria monocytogenes, Salmonella typhimurium or Clostridium difficile. In an embodiment, the target molecule is selected from the group consisting of small inorganic molecule, small organic molecule, metal ion, biomolecule, toxin, biopolymer, optionally nucleic acid, carbohydrate, lipid, peptide, protein, optionally glutamate dehydrogenase.

In an embodiment, the oligonucleotides include a primer region to allow for amplification and a random single stranded DNA sequence domain of about 20 to about 80 nucleotides, optionally about 40 nucleotides, and wherein the primer region comprises sequences of SEQ ID NOs: 2 or 63, and 64.

In another aspect, there is also provided is a circular DNA aptamer that binds to C. difficile glutamate dehydrogenase (GDH), wherein the circular DNA aptamer comprises a sequence of SEQ ID NO: 6, 7, 14, or 16, or a functional fragment or modified derivative thereof.

In another aspect, there is also provided is an aptamer probe comprising the circular DNA aptamer of that binds to C. difficile glutamate dehydrogenase (GDH), wherein the circular DNA aptamer comprises a sequence of SEQ ID NO: 6, 7, 14, or 16, or a functional fragment or modified derivative thereof, and a detectable label, optionally the detectable label is a fluorescent moiety, optionally the fluorescent moiety is a fluorophore.

In another aspect, there is also provided a biosensor system comprising:
a) a circular DNA aptamer attached directly or indirectly to a solid support, wherein the circular DNA aptamer comprises a circular DNA aptamer comprising a sequence of SEQ ID NO: 6, 7, 14, or 16, or a functional fragment or modified derivative thereof.

In an embodiment, the circular DNA aptamer indirectly via GDH attached to the solid support.

In an embodiment, the biosensor system further comprises components for detecting a signal through rolling circle amplification (RCA) of the circular DNA aptamer in a test sample.

In another aspect, there is also provided a method for detecting the presence of a target C. difficile glutamate dehydrogenase (GDH) in a test sample, comprising:
a) contacting the test sample with a circular DNA aptamer to form a mixture, wherein the circular DNA aptamer is capable of binding to the target C. difficile glutamate dehydrogenase to form a complex;
b) separating the complex from the mixture; and
c) detecting a signal from the complex through rolling circle amplification (RCA) of the circular DNA aptamer,
wherein detection of a signal indicates the presence of the target molecule in the test sample and the lack of signal indicates the absence of the target molecule, and
wherein the circular aptamer comprises a circular DNA aptamer comprising the sequence of SEQ ID NO: 6, 7, 14, or 16, or a functional fragment or modified derivative thereof.

In another aspect, there is also provided a method of detecting C. difficile infection in a subject comprising:
a) testing a sample from the subject for the presence of C. difficile GDH by a method of detecting the presence of a C. difficile glutamate dehydrogenase (GDH) in a test sample described herein; and
b) if GDH is present, further comprising testing the sample for the presence of C difficile toxins A and B,
wherein the presence of GDH and the presence of toxins A and B indicate that the subject has a C. difficile infection.

In an embodiment, the testing for the presence of C. difficile toxins A and B is selected from the group consisting of cell cytotoxicity neutralization assay, toxin enzyme immunoassay or detection of toxin genes using PCR. In an embodiment, the method further comprises treating the subject for C. difficile infection if GDH and toxins are present.

In another aspect, there is also provided a kit for detecting C. difficile GDH, wherein the kit comprises a circular DNA aptamer described herein and instructions for use of the kit.

In another aspect, there is also provided a kit for detecting C. difficile GDH, wherein the kit comprises the components required for a method of detecting C. difficile described herein and instructions for use of the kit.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should be given the broadest interpretation consistent with the description as a whole.

### DRAWINGS

The embodiments of the disclosure will now be described in greater detail with reference to the attached drawings in which:

FIG. 1a shows in vitro selection of circular aptamers (captamers) for GDH. There are 5 steps: library circularization, counter selection with bare magnetic beads, positive selection with rGDH-coated magnetic beads, elution of circular DNAs bound to rGDH-coated magnetic beads, and DNA amplification.

FIG. 1b shows the sequences of the original DNA library and the two featured captamers isolated from the library. PBS: primer-binding site.

FIG. 2a is a schematic of the pulldown assay that determines the dissociation constants (K_{d}) of the radioactively labeled linear and circular aptamers 2 and 4 for rGDH.

FIG. 2b shows the data obtained when it was applied to determine the dissociation constants (K_{d}) of the radioactively labeled linear and circular aptamers 2 and 4 for rGDH. The relative radioactivity in the DNA band is calculated by taking the highest radioactivity reading in each series as 1. The binding data was fitted with the Origin software to a saturation 1:1 binding curve using nonlinear regression.

FIG. 2c is a schematic of the dot blotting assay for binding of rGDH by the radioactive linear and circular aptamers 2 and 4.

FIG. 2d shows the results obtained with binding of rGDH by the radioactive linear and circular aptamers 2 and 4.

FIG. 3a shows competitive binding assays using ³²P-labeled Capt-2 and nonradioactive Lapt-2.

FIG. 3b shows competitive binding assays using ³²P-labeled Lapt-2 and Capt-4.

FIG. 3c shows sandwich dot blotting assays using immobilized Lapt-2 as a capture agent and ³²P-labeled Capt-4 as a detection agent. Four rows represent four repeats.

FIG. 4a is a schematic of the reaction for filter binding assays using ³²P-labeled Capt-4 and different amounts of immobilized nGDH.

FIG. 4b shows radioimaging data for filter binding assays using ³²P-labeled Capt-4 and different amounts of immobilized nGDH.

FIG. 4c shows binding curve measured by filter binding assay for Capt-4 to nGDH.

FIG. 5a is a schematic representation of the Captamer-RCA biosensing

FIG. 5b shows time-dependent fluorescence upon incubation of SYBR Gold with RCA products obtained with varying nGDH concentrations.

FIG. 5c shows fluorescence intensities of a proposed biosensor from four healthy persons (N) and four CDI patients (P) using a Genie II instrument for fluorescence reading.

FIG. 6a is a schematic illustration of the preparation and circularization of the DNA library DL1 by T4 DNA ligase-mediated ligation (p: 5′-phosphate).

FIG. 6b shows circular DNA amplification by PCR. PCR1 uses FP1 and RP1 as primers, and PCR2 uses FP1 and RP2 as primers. RP2 contains an internal C3 spacer and All tail at the 5′ end. The spacer prevents the poly-A tail from being amplified, making the non-aptamer-coding strand 12-nt longer than the aptamer-coding strand. The aptamer-coding strand was then purified by 10% dPAGE.

FIG. 7 shows SELEX progress. The percent DNA bound to rGDH-coated magnetic beads, measured by RT-PCR, was calculated as the amount of bound DNA divided by the total input DNA. The time of incubation between the DNA pool and the beads was 30 min for rounds 1-7, and was then reduced to 15 min for rounds 8-12.

FIG. 8 shows results of a binding assay that identifies potential captamer sequences. The signal-to-background ratio (S/B) was defined as S/B=I₁/I₂, where I₁ and I₀ were the measured radioactive signal on beads coated with and without rGDH. The scrambled sequences of Capt-2 and Capt-4 were generated using the Sequence Manipulation Suite: Shuffle DNA web based program (http://www.bioinformatics.org/sms2/shuffle_dna.html).

FIG. 9 shows predicted secondary structures of Capt-2 and Lapt-2 using the m-fold program. Folding conditions: 25° C., 150 mM NaCl, 15 mM MgCl₂. Letters in grey are nucleotides in the original random-sequence domain, those in black are nucleotides in the primer domains. Common elements in circular and linear structures are annotated L1 (L: loop), L2, B1 (bulge) and B2.

FIG. 10 shows predicted secondary structures of Capt-4 and Lapt-4 using the m-fold program. Folding conditions: 25° C., 150 mM NaCl, 15 mM MgCl₂. Letters in grey are nucleotides in the original random-sequence domain, those in black are nucleotides in the primer domains. Common structural elements are annotated L1 and L2. The distinct structural element within the random-sequence domain in Capt-4 is labeled as X1.

FIG. 11 shows results of competitive binding assays using ³²P-labeled Anti-G1T1, Anti-G1, Anti-G3 and Anti-G7. Anti-G1, Anti-G3 and Anti-G7 were linear aptamers previously selected from the linear library. Anti-G1 was the linear form of Capt-2 discovered and provided in this disclosure (SEQ ID NO: 6). Anti-G1T1 is a shortened version of Anti-G1 with full activity. These aptamers were set up to compete with each other for binding to rGDH using the pull-down assay. Lanes B, D, G and J show that the amount of radioactive Anti-G1T1, Anti-G1, Anti-G3 and Anti-G7 pulled down by rGDH-coated NiMBs when each radioactive aptamer was individually incubated with the beads, while Lanes E, H, K depict the amount of Anti-G1T1/Anti-G1, Anti-G1T1/Anti-G3 and Anti-G1T1/Anti-G7 pulled down when the named pair were combined and incubated with the beads. The significant reduction of both Anti-G1T1 and its paired aptamer in these lanes is consistent with the expected competitive binding between Anti-G1T1 and each of the paired aptamer. The results clearly show that all three linear aptamers recognize the same site on rGDH. Experimental details: The protocol is similar to the one described in Example 1G for the use of different aptamers named above.

FIG. 12 shows dot blotting analysis using [³²P]-labeled Capt-4 and different protein targets. Experimental details: The protocol is similar to the one described in Example 1E except for: A total of 2 μL of BSA (1.5 μg/μL), 2 μL of TcdA (1.5 μg/μL), 1.8 μL of TcdB (1.7 μg/μL) and 4.6 μL of nGDH (0.65 μg/uL) were spotted onto a nitrocellulose membrane.

FIG. 13 shows chemical stability of Lapt-4 and Capt-4 in biological faeces. Experimental details: A total of 50 nM γ-[³²P]-labeled Lapt-4 or Capt-4 was incubated in 10 μL of unformed faecal specimens at RT for periods up to 3 h. Afterward, the mixtures were heated to 90° C. for 10 min and analyzed by 10% dPAGE.

FIG. 14 shows functional stability of Lapt-4 and Capt-4 in biological faeces. Experimental details: A total of 50 nM γ-[P]-labeled Lapt-4 or Capt-4 was incubated in 20 μL of 1× binding buffer containing 10 μL of unformed faecal specimens at RT for periods up to 5 h. Following incubation, the mixtures were added to the nGDH-coated nitrocellulose membrane and incubated for 20 min. After washing with 1× binding buffer three times, these paper samples were dried at RT prior to imaging.

FIG. 15 shows schematics of nGDH-induced inhibition effect on the RCA reaction, and analysis of the RCA products (RP) in the presence of various nGDH concentrations. Experimental details: The protocol is similar to the one described in Experiment Section 9 above except for: no heating step was included.

FIG. 16a shows analysis of RCA products (RP) by 0.6% agarose gel electrophoresis.

FIG. 16b shows specificity test of a bioassay of this disclosure.

FIG. 17 shows time-dependent fluorescence upon incubation of SYBR Gold with RCA products obtained with varying nGDH levels using Genie II portable fluorimeter platform.

FIG. 18 shows fluorescent results of the RCA assay system obtained using varying nGDH levels in pure buffer and spiked in faeces.

FIG. 19 shows real-time fluorescence responses of the biosensor from four healthy persons (N) and CDI patients (P) using Genie II portable fluorimeter platform.

### DETAILED DESCRIPTION

Although many SELEX studies have been described for diverse targets, to the inventors' knowledge, all these experiments used linear nucleic acid libraries. A circular DNA library was used for the selection of a DNA-cleaving DNAzyme [2d], however, aptamer selection with nucleic acid libraries of circular topology has never been attempted. This disclosure herein describes the isolation of circular aptamers (simplified as captamers) directly from a circular DNA library.

There are several advantages associated with selecting captamers. First, previous studies have shown that circular DNA aptamers, produced via circularization of linear DNA aptamers, offer enhanced biological stability (as they are resistant to exonuclease degradation), making them highly desirable for applications that involve real biological samples [4]. Second, placing aptamers in a circular form creates opportunities for the design of biosensors that incorporate “rolling circle amplification (RCA)” as a signal amplification mechanism, as this technique involves copying a circular template by a DNA polymerase [4,5]. Although a linear aptamer can be redesigned into a captamer, additional nucleotides will have to be carefully incorporated into new constructs to minimize the impact on the activity of the aptamer imposed by the circularization [6]. Selection of DNA aptamers directly from a circular DNA library should provide optimal captamers for such applications. Importantly, selecting aptamers using circular DNA libraries offers a unique opportunity to search for aptamers with novel properties that can only be provided with the circular topology.

As shown in this disclosure, selecting aptamers from a circular DNA library can generate circular DNA aptamers with properties considerably different from the aptamers selected from a linear library.

I. Definitions

Unless otherwise indicated, the definitions and embodiments described in this and other sections are intended to be applicable to all embodiments and aspects of the present disclosure herein described for which they are suitable as would be understood by a person skilled in the art.

The term “aptamer” as used herein refers to a short, chemically synthesized nucleic acid molecule or oligonucleotide which fold into specific three-dimensional (3D) structures that interact with a target molecule with dissociation constants in the pico- to nano-molar range. In general, aptamers may be single-stranded DNA or RNA, and may include modified nucleotides, modified backbones, for example in a peptide nucleic acid (PNA), and/or nucleotide derivatives.

The term “nucleic acid aptamer” and its derivatives, as used herein, are intended to refer to an aptamer comprising a nucleic acid molecule described herein, such as an unmodified DNA or RNA or modified DNA or RNA, or modified backbone nucleic acids, such as PNA, that are derived from the DNA base sequence.

The term “DNA aptamer” as used herein refers to an aptamer comprising DNA or comprising modified backbone nucleic acids, such as PNA, that are derived from the DNA base sequence.

Aptamers can be in linear or circular form. Circular aptamer is also known as captamer, which has circular topology. Circular nucleic acid aptamers aptamer can be produced via circularization (i.e. end-to-end ligation) of linear nucleic acid aptamers. As well, a plurality of circular nucleic acid aptamers (i.e. circular nucleic acid aptamer library or pool), for example a plurality of circular DNA aptamers, can be created from a plurality of linear DNA molecules (i.e. linear DNA library or pool) as described in Example 1C, the first step of which is circularization of linear DNA molecules. Circularization of linear nucleic acid molecules such as DNA molecules may involve, for example, a ligation template which is a nucleic acid oligonucleotide such as DNA oligonucleotide that binds the 5′-end and 3′-end of a linear molecule to create a duplex structure for a ligase such as T4 DNA ligase mediated ligation. The resulting pool of circular nucleic acid molecules such as DNA molecules can then be used in selection, counter-selection, and/or amplification for generating circular nucleic acid aptamers such as DNA aptamers. Circular nucleic acid aptamers such as DNA aptamers are useful in biosensors or biosensor systems that incorporate rolling circle amplification (RCA) as a signal amplification mechanism, as this technique involves copying a circular template by a polymerase such as a DNA polymerase. In an embodiment, circular DNA aptamer comprising a sequence of SEQ ID NO: 6, 7, 14, or 16, or a functional fragment or modified derivative thereof is a RCA template. In an embodiment, circular DNA aptamer comprising a sequence of SEQ ID NO: 6, or a functional fragment or modified derivative thereof is a RCA template. In an embodiment, circular DNA aptamer comprising a sequence of SEQ ID NO: 7, or a functional fragment or modified derivative thereof is a RCA template. In an embodiment, circular DNA aptamer comprising a sequence of SEQ ID NO: 14, or a functional fragment or modified derivative thereof is a RCA template. In an embodiment, circular DNA aptamer comprising a sequence of SEQ ID NO: 16, or a functional fragment or modified derivative thereof is a RCA template.

The term “rolling circle amplification” or “RCA” as used herein refers to a unidirectional nucleic acid replication that can rapidly synthesize multiple copies of circular nucleic acid molecules. In an embodiment, rolling circle amplification is an isothermal enzymatic process where a short DNA or RNA primer is amplified to form a long single stranded DNA or RNA using a circular nucleic acid template and an appropriate DNA or RNA polymerase. The product of this process is a concatemer containing ten to thousands of tandem repeats that are complementary to the circular template. A method of RCA comprises: annealing a primer to a circular template where the circular template comprises a region complementary to the primer and an AC rich nucleotide region; amplifying the circular template under conditions that allow rolling circle amplification.

Rolling circle amplification conditions are known in the art. For example, rolling circle amplification occurs in the presence of a polymerase that possesses both strand displacement ability and high processivity in the presence of template, primer and nucleotides. In an embodiment, rolling circle amplification conditions comprise temperatures of from about 20° C. to about 35° C., or about 30° C., a reaction time sufficient for the generation of detectable amounts of amplicon and performing the reaction in a buffer. In an embodiment, the rolling circle amplification conditions comprise the presence of phi29-, Bst-, or Vent exo-DNA polymerase. In an embodiment, the rolling circle amplification conditions comprise the presence of phi29-DNA polymerase.

A person skilled in the art would understand that there are numerous ways to detect the presence of single stranded DNA or RNA molecules in a test sample after rolling circle amplification and includes, without limitation, radioactive, electrochemical, spectroscopic and colorimetric detection and/or quantification. For example, the generated DNA or RNA molecules can be labeled radioactively or detected by hybridizing with a complementary nucleic acid molecule, optionally coupled to a detectable labeled.

The term “nucleic acid molecule” and its derivatives, as used herein, are intended to include unmodified DNA or RNA or modified DNA or RNA. The nucleic acid molecules of the disclosure may contain one or more modified bases or DNA or RNA backbones modified for stability or for other reasons. “Modified” bases include, for example, tritiated bases and unusual bases such as inosine. A variety of modifications can be made to DNA and RNA; thus “nucleic acid molecule”, “DNA molecule”, and “RNA molecule” embrace chemically, enzymatically, or metabolically modified forms. The term “polynucleotide” shall have a corresponding meaning. The term “oligonucleotide” refers to a nucleic acid molecule having a sequence of at least about 13 nucleotides residues and up to about 220 nucleotide residues. Examples of modified nucleotides which can be used to generate the nucleic acids disclosed herein include xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8 amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil and 5-trifluoro cytosine. Alternatively, the nucleic acid molecules can be produced biologically using an expression vector.

Another example of a modification is to include modified phosphorous or oxygen heteroatoms in the phosphate backbone, short chain alkyl or cycloalkyl intersugar linkages or short chain heteroatomic or heterocyclic intersugar linkages in the nucleic acid molecules. For example, the nucleic acid sequences may contain phosphorothioates, phosphotriesters, methyl phosphonates, and phosphorodithioates.

A further example of an analog of a nucleic acid molecule of the disclosure is a peptide nucleic acid (PNA) wherein the deoxyribose (or ribose) phosphate backbone in the DNA (or RNA), is replaced with a polyamide backbone which is similar to that found in peptides (P. E. Nielsen, et al Science 1991, 254, 1497). PNA analogs have been shown to be resistant to degradation by enzymes and to have extended lives in vivo and in vitro. PNAs also bind stronger to a complementary DNA sequence due to the lack of charge repulsion between the PNA strand and the DNA strand. Other nucleic acid analogs may contain nucleotides containing polymer backbones, cyclic backbones, or acyclic backbones. For example, the nucleotides may have morpholino backbone structures (U.S. Pat. No. 5,034,506).

The term “target” or “target molecule” as used herein may refer to any agent, including, but not limited to, a small inorganic molecule, small organic molecule, metal ion, biomolecule, toxin, biopolymer (such as a nucleic acid, carbohydrate, lipid, peptide, protein), cell, tissue, microorganism, virus and pathogen, for which one would like to sense or detect. In an embodiment, the analyte is either isolated from a natural source or is synthetic. The analyte may be a single compound or a class of compounds, such as a class of compounds that share structural or functional features. The term analyte also includes combinations (e.g. mixtures) of compounds or agents such as, but not limited to, combinatorial libraries and samples from an organism or a natural environment. In an embodiment, the analyte comprises a microorganism target.

The term a “microorganism target” as used herein may be a molecule, compound or substance that is present in or on a microorganism or is generated, excreted, secreted or metabolized by a microorganism. In an embodiment, the microorganism target is present in the extracellular matrix of a microorganism. In another embodiment, the microorganism target is present in the intracellular matrix of a microorganism. In another embodiment, the microorganism target comprises a protein, a nucleic acid, a small molecule, extracellular matrix, intracellular matrix, a cell of the microorganism, or any combination thereof. In an embodiment, the microorganism target is a crude or purified extracellular matrix or a crude or purified intracellular matrix. In another embodiment, the microorganism target is specific to a particular species or strain of microorganism.

The term “microorganism” as used herein may refer to a microscopic organism that comprises either a single cell or a cluster of single cells including, but not limited to, bacteria, fungi, archaea, protists, algae, plankton and planarian. In an embodiment, the microorganism is a bacterium. In an embodiment, the microorganism is a pathogenic bacterium (for example, a bacterium that causes bacterial infection), such as Escherichia coli O157:H7, Listeria monocytogenes, Salmonella typhimurium or Clostridium difficile.

As used herein, “test sample” refers to a sample in which the presence or amount of a target or target molecule is unknown and to be determined in an assay, preferably a diagnostic test. The test sample may be a “biological sample” comprising cellular and non-cellular material, including, but not limited to, tissue samples, urine, blood, serum, other bodily fluids, and excrement, such as a stool (i.e. faeces) sample from a subject, or an “environmental sample” obtained from water, soil or air. In an embodiment, the test sample target is GDH of C. difficile.

The term “treatment or treating” as used herein means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable.

As used herein, the term “random nucleotide domain” refers to a random sequence domain of, for example, about 20 to about 80 nucleotides, which is a part of a nucleic acid molecule in a random-sequence library of nucleic acid molecules for aptamer selection. In some instance, the random sequence domain is about 40 nucleotides.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this disclosure, the singular forms “a”, “an” and “the” include plural references unless the content clearly dictates otherwise.

In embodiments comprising an “additional” or “second” component, such as an additional or second component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The term “subject” as used herein includes all members of the animal kingdom including mammals such as a mouse, a rat, a dog, and a human.

II. Methods of Identifying or Producing A Circular Aptamer

In a broad aspect, herein provided is a method of identifying or producing a circular nucleic acid aptamer capable of binding to a target molecule, wherein said method comprises:
a) incubating or contacting a plurality of circular nucleic acid molecules in the presence of the target molecule;
b) collecting the plurality of circular nucleic acid molecules that bind to the target molecule; and
c) amplifying the plurality of circular nucleic acid molecules of b) to yield a mixture of a plurality of circular nucleic acid aptamers enriched in nucleic acid sequences that are capable of binding to the target molecule.

In an embodiment, the circular nucleic acid aptamer is a circular DNA aptamer. In an embodiment, the target is immobilized. In an embodiment, the method further comprises testing for binding to the target molecule. In an embodiment, the oligonucleotides include 5′-end primer region and a 3′-end primer region to allow for amplification and a random single stranded DNA sequence domain of about 20 to about 80 nucleotides, optionally about 40 nucleotides. A person skilled in the art can readily recognize the appropriate sequence for the primer regions for use in Systematic Evolution of Ligands by Exponential Enrichment (SELEX). In an embodiment, the sequence for the primer regions are compatible with SELEX.

In a further aspect of the present disclosure, the inventors provide a method for identifying or producing an aptamer capable of binding to a target molecule using circular DNA molecules. Accordingly, herein provided is a method of identifying or producing an aptamer capable of binding to a target molecule, wherein the method comprises:
a) providing a plurality of circular nucleic acid molecules, wherein the plurality of circular nucleic acid molecules comprises oligonucleotides having at least one random nucleotide domain flanked by a 5′-end primer region and a 3′-end primer region;
b) contacting the plurality of circular nucleic acid molecules with a bare solid support;
c) collecting unbound circular nucleic acid molecules;
d) contacting the unbound circular nucleic acid molecules with a solid support coated with the target molecule to form a complex comprising bound circular nucleic acid molecules;
e) optionally washing the complex;
f) eluting the bound circular nucleic acid molecules from the complex; and
g) amplifying the eluted circular nucleic acid molecules by polymerase chain reaction (PCR).

In an embodiment, the contacting comprises contacting in a binding buffer. In an embodiment, the method further comprises repeating steps b) to g) or d) to g) for one to nineteen times, optionally for six to eleven times, with the amplified eluted circular nucleic acid molecules of g) as the plurality of circular nucleic acid molecules in step b) or the unbound circular nucleic acid molecules of d). In an embodiment, the method further comprises repeating steps b) to g) for one to nineteen times, optionally for six to eleven times, with the amplified eluted circular nucleic acid molecules of g) as the plurality of circular nucleic acid molecules in step b). In an embodiment, the method further comprises repeating steps d) to g) for one to nineteen times, optionally for six to eleven times, with the amplified eluted circular nucleic acid molecules of g) as the unbound circular nucleic acid molecules of d). In an embodiment, step b) comprises contacting the plurality of nucleic acid molecules with a solid support coated with an off-target molecule. In an embodiment, the method further comprises repeating steps b) to g) for eleven times. In an embodiment, the oligonucleotides include a primer region to allow for amplification and a random single stranded nucleic acid sequence domain of about 20 to about 80 nucleotides, optionally about 40 nucleotides. In an embodiment, the primer region comprises sequence of SEQ ID NOs: 2 or 63, and 64. In an embodiment, the primer region comprises sequence of SEQ ID NOs: 2 and 64. In an embodiment, the primer region comprises sequence of SEQ ID NOs: 63 and 64. In an embodiment, the circular nucleic acid molecules are circular DNA molecules.

In an embodiment, the method further comprises repeating e) washing the complex for one or two times. In an embodiment, the washing comprises washing with a binding buffer. In an embodiment, the binding buffer comprises a physiological buffer. In an embodiment, the binding buffer comprises HEPES, NaCl, MgCl₂, KCl and Tween-20. In a specific embodiment, the binding buffer comprises 50 mM HEPES, 150 mM NaCl, 10 mM MgCl₂, 5 mM KCl and 0.02% Tween-20. In an embodiment, step f) eluting comprises denaturing the circular nucleic acid molecules, optionally wherein the denaturing comprises heating or urea treatment, optionally 10M urea.

In an embodiment, providing a plurality of circular nucleic acid molecules comprises preparing a plurality of circular nucleic acid molecules by circularizing a plurality of linear nucleic molecules. In an embodiment, the circularizing a plurality of linear nucleic acid molecules comprises template-assisted ligation. In an embodiment, the circular nucleic acid molecules are circular DNA molecules.

In an embodiment, the solid support comprises nitrocellulose filter disc or magnetic beads. In an embodiment, the nitrocellulose filter disc comprises a pore size 0.45 or 0.22 um. In an embodiment, the solid support comprises magnetic beads. In an embodiment, the solid support comprises magnetic beads coated with a metal. In an embodiment, the solid support comprises magnetic beads comprising silica, polystyrene, or agarose, coated with nickel, cobalt, copper, iron, zinc, or aluminum. In an embodiment, the magnetic beads comprises silica, polystyrene, or agarose. In an embodiment, the metal comprises nickel, cobalt, copper, iron, zinc, or aluminum. In an embodiment, the solid support comprises Nickel Nitrilotriacetic acid magnetic beads (NiMBs).

In an embodiment, the target molecule is a cell, tissue, microorganism, virus and pathogen. In an embodiment, the target molecule is a microorganism target or a target molecule present on or generated from a microorganism or a virus. In an embodiment, target molecule is a virus. In an embodiment, the target molecule is a pathogen. In an embodiment, the target molecule is a microorganism. In an embodiment, the microorganism is selected from the group consisting of a bacteria, fungi, archaea, protists, algae, plankton and planarian. In an embodiment, the microorganism is a pathogenic bacterium. In an embodiment, the pathogenic bacterium is selected from the group consisting of Escherichia coli O157:H7, Listeria monocytogenes, Salmonella typhimurium or Clostridium difficile.

In an embodiment, the target molecule is selected from the group consisting of small inorganic molecule, small organic molecule, metal ion, biomolecule, toxin, biopolymer, optionally nucleic acid, carbohydrate, lipid, peptide, protein, optionally glutamate dehydrogenase.

III. Aptamers, Probes and Biosensor Systems of the Disclosure

The term “GDH” or “glutamate dehydrogenase” as used herein refers to GDH from any source or organism. In an embodiment, the GDH is C. difficile GDH having a protein sequence as set out in Genbank Accession No. AAA62756.1 (SEQ ID NO: 18).

Accordingly, the disclosure provides a circular DNA aptamer that interacts with and binds to GDH through structural recognition. In an embodiment, the development of an aptamer that binds GDH is produced through Systematic Evolution of Ligands by EXponential enrichment (SELEX) technology.

The inventors identified in the screening circular DNA aptamers that bind to GDH, namely capt-2 (SEQ ID: 6) and capt-4 (SEQ ID NO: 7), which have the random sequence domain of SEQ ID NO: 14 and SEQ ID NO: 16, respectively. In an embodiment, the circular aptamer that binds to C. difficile GDH comprises or consists of a sequence of SEQ ID NOS: 6, 7, 14, or 16, or a functional fragment or modified derivative thereof. In an embodiment, the circular aptamer that binds to C. difficile GDH comprises or consists of a sequence of SEQ ID NO: 6 or 14, or a functional fragment or modified derivative thereof. In an embodiment, the circular aptamer that binds to C. difficile GDH comprises or consists of a sequence of SEQ ID NO: 6, or a functional fragment or modified derivative thereof. In an embodiment, the circular aptamer that binds to C. difficile GDH comprises or consists of a sequence of SEQ ID NO: 14, or a functional fragment or modified derivative thereof. In an embodiment, the circular aptamer that binds to C. difficile GDH comprises or consists of a sequence of SEQ ID NO: 7 or 16, or a functional fragment or modified derivative thereof. In an embodiment, the aptamer that binds to C. difficile GDH comprises or consists of a sequence of SEQ ID NO: 7, or a functional fragment or modified derivative thereof. In an embodiment, the aptamer that binds to C. difficile GDH comprises or consists of a sequence of SEQ ID NO: 16, or a functional fragment or modified derivative thereof. The term “functional fragment” as used herein refers to the ability of the fragment to act as an aptamer to bind to GDH and change conformation upon the binding. The circular DNA aptamer sequence of SEQ ID NO: 16 can have additional surrounding sequence at the 5′ and 3′ ends (see Table 4). In an embodiment, the aptamer that binds to C. difficile GDH comprises a sequence of SEQ ID NO: 16, further comprises a sequence of any one of SEQ ID NO: 21-41 at the 5′ end, and a sequence of any one of SEQ ID NO: 42-62 at the 3′ end.

The term “aptamer probe” as used herein refers to an aptamer coupled to a detectable label.

In another aspect, herein provided is an aptamer probe that comprises an aptamer disclosed herein and a detectable label. In one embodiment, the detectable label comprises a fluorescent, a colorimetric or other optical probe or electrochemical moiety. In a particular embodiment, the detectable label is a fluorescent moiety, optionally a fluorophore. The fluorophore may be any fluorophore, such as a chemical fluorophore, for example, one selected from fluorescein, rhodamine, coumarin, cyanine or derivatives thereof. In one embodiment, the detectable label is FAM or Cy5. The selection of the fluorophore is based upon one or more parameters including, but not limited to, (i) maximum excitation and emission wavelength, (ii) extinction coefficient, (iii) quantum yield, (iv) lifetime, (v) stokes shift, (vi) polarity of the fluorophore and (vii) size.

In an embodiment, the circular DNA aptamer probe comprises or consists of the sequence of SEQ ID NOS: 6, 7, 14, or 16, or a functional fragment or modified derivative thereof. In an embodiment, the circular DNA aptamer probe comprises or consists of the sequence of SEQ ID NO: 6, or a functional fragment or modified derivative thereof. In an embodiment, the circular DNA aptamer probe comprises or consists of the sequence of SEQ ID NO: 7, or a functional fragment or modified derivative thereof. In an embodiment, the circular DNA aptamer probe comprises or consists of the sequence of SEQ ID NO: 14, or a functional fragment or modified derivative thereof. In an embodiment, the circular DNA aptamer probe comprises or consists of the sequence of SEQ ID NO: 16, or a functional fragment or modified derivative thereof.

In another aspect, herein provided is a biosensor system comprising:
a) a circular DNA aptamer attached directly or indirectly to a solid support,
wherein the circular DNA aptamer comprises a circular DNA aptamer comprising a sequence of SEQ ID NO: 6, 7, 14, or 16, or a functional fragment or modified derivative thereof.

In an embodiment, the circular DNA aptamer indirectly via GDH attached to the solid support. In an embodiment, the circular DNA aptamer comprises a circular DNA aptamer comprising a sequence of SEQ ID NO: 6, or a functional fragment or modified derivative thereof. In an embodiment, the circular DNA aptamer comprises a circular DNA aptamer comprising a sequence of SEQ ID NO: 7, or a functional fragment or modified derivative thereof. In an embodiment, the circular DNA aptamer comprises a circular DNA aptamer comprising a sequence of SEQ ID NO: 14, or a functional fragment or modified derivative thereof. In an embodiment, the circular DNA aptamer comprises a circular DNA aptamer comprising a sequence of SEQ ID NO: 16, or a functional fragment or modified derivative thereof.

In an embodiment, the biosensor system further comprises components for detecting a signal through rolling circle amplification (RCA) of the circular DNA aptamer in a test sample. In an embodiment, the components for RCA comprise a DNA polymerase. In an embodiment, the components for RCA comprise phi29-, Bst-, or Vent exo-DNA polymerase. In an embodiment, the components for RCA comprise phi29-DNA polymerase. In an embodiment, detection of a signal through RCA indicates the presence of the target molecule. In an embodiment, the lack of detection of a signal through RCA indicates the absence of the target molecule. In an embodiment, circular DNA aptamer further comprises a detectable label. In an embodiment, the circular DNA aptamer is capable of binding to the target molecule to form an aptamer-target molecule complex.

In an embodiment, the solid support comprises nitrocellulose filter disc or magnetic beads. In an embodiment, the nitrocellulose filter disc comprises a pore size 0.45 or 0.22 um. In an embodiment, the solid support comprises magnetic beads. In an embodiment, the solid support comprises magnetic beads coated with a metal. In an embodiment, the solid support comprises magnetic beads comprising silica, polystyrene, or agarose, coated with nickel, cobalt, copper, iron, zinc, or aluminum. In an embodiment, the magnetic beads comprises silica, polystyrene, or agarose. In an embodiment, the metal comprises nickel, cobalt, copper, iron, zinc, or aluminum. In an embodiment, the solid support comprises Nickel Nitrilotriacetic acid magnetic beads (NiMBs).

In an embodiment, the circular DNA aptamer is an aptamer probe. The aptamer probes disclosed herein are also useful as part of a typical signaling structure-switching nucleic acid aptamer (FQDNA) biosensor system.

The term “structure-switching nucleic acid aptamers” or “reporter nucleic acid aptamers” as used herein refers to aptamer-based reporters that function by switching structures from a DNA/DNA or RNA/RNA complex to a DNA/target or RNA/target complex.

This general assay design is based on the change in conformation from a DNA/DNA duplex to a DNA/target complex. In this assay, an oligonucleotide is generated that contains an aptamer flanked by a primer region. A fluorophore-labeled oligonucleotide (FDNA) hybridizes to the primer region, while the quencher-labeled oligonucleotide (QDNA) hybridizes to the aptamer region. In the absence of target, this DNA/DNA duplex will be weakly fluorescent as a result of the close proximity of the quencher and fluorophore. Upon introduction of target, the aptamer forms a DNA/target complex, displacing the QDNA and producing a large increase in fluorescence intensity. The magnitude of signal generation is dependent upon the concentration of target added.

Accordingly, there is provided in the disclosure a biosensor system comprising an aptamer probe disclosed herein in association with a quencher-oligonucleotide that quenches the detectable label; wherein the aptamer changes conformation upon binding GDH and results in release from the quencher such that the label is able to be detected. In some embodiments, the quencher-oligonucleotide is a DNA that hybridizes with the aptamer probe in the absence of GDH.

In an embodiment, the quencher molecule is selected from dimethylaminoazobenzenesulfonic acid (dabcyl) and fluorescence resonance energy transfer (FRET or blackhole) quenchers and derivatives thereof.

In yet another aspect of the disclosure, a structure-switching signaling aptamer-based biosensor system for real-time, sensitive and selective detection of GDH is disclosed. Accordingly, the present disclosure provides a biosensor system comprising an aptamer probe disclosed herein associated with a quencher molecule; wherein the aptamer changes conformation upon binding to GDH and results in displacement of the quencher from the aptamer probe.

A quencher molecule is a substance with no native fluorescence and that absorbs the excitation energy from a fluorophore and dissipates the energy as heat, with no emission of fluorescence. Thus, when the fluorophore and quencher are close in proximity, the fluorophore's emission is suppressed.

Also provided herein is another biosensor system comprising:
a) a circular DNA aptamer probe; and
b) a nanomaterial;
wherein the circular DNA aptamer probe is adsorbed on the nanomaterial;
wherein the circular DNA aptamer changes conformation upon binding GDH and results in desorption from the nanomaterial; and
wherein the circular DNA aptamer probe comprises a circular DNA aptamer comprising a sequence of SEQ ID NO: 6, 7, 14, or 16, or a functional fragment or modified derivative thereof.

In an embodiment, the circular DNA aptamer comprises a circular DNA aptamer comprising a sequence of SEQ ID NO: 6, or a functional fragment or modified derivative thereof. In an embodiment, the circular DNA aptamer comprises a circular DNA aptamer comprising a sequence of SEQ ID NO: 7, or a functional fragment or modified derivative thereof. In an embodiment, the circular DNA aptamer comprises a circular DNA aptamer comprising a sequence of SEQ ID NO: 14, or a functional fragment or modified derivative thereof. In an embodiment, the circular DNA aptamer comprises a circular DNA aptamer comprising a sequence of SEQ ID NO: 16, or a functional fragment or modified derivative thereof.

In an embodiment, the nanomaterial acts as a quencher molecule. In an embodiment, the nanomaterial may be any nanomaterial that is able to have nonspecific DNA binding affinity and allow target-induced binding, such as reduced graphene oxide (RGO) or metal particles, such as gold or platinum particles.

In an embodiment, the nanomaterial is reduced graphene oxide (RGO). In some embodiments, reduced graphene oxide is produced by reducing an aqueous solution of graphene oxide, prepared, for example as described in M. Liu, et al. ACS Nano 2012, 6, 3142-3151, with a reducing agent, such as ascorbic acid and ammonia, followed by heating, for example to about 80° C. to about 100° C. for about 3 to about 10 minutes. Cooling this solution to room temperature provides a stably dispersed RGO solution.

In an embodiment, the biosensor system is comprised of a fluorometric circular DNA aptamer probe adsorbed to the surface of reduced graphene oxide (RGO), wherein conformational changes in the circular DNA aptamer probe induced by binding to GDH result in desorption of the circular DNA aptamer probe from the RGO, to provide RGO and a GDH-aptamer probe complex that is detectable fluorometrically. In an embodiment, conformational changes in the circular DNA aptamer or circular DNA aptamer probe induced by binding to GDH result in a RCA reaction. In an embodiment, the RCA reaction produces an amplified signal. In an embodiment, the biosensor system further comprises components for detecting a signal through rolling circle amplification (RCA) of the circular DNA aptamer or circular DNA aptamer probe in a test sample. In an embodiment, the components for RCA comprise a DNA polymerase. In an embodiment, the components for RCA comprise phi29-, Bst-, or Vent exo-DNA polymerase. In an embodiment, the components for RCA comprise phi29-DNA polymerase.

In an embodiment, the nanomaterial, such as RGO is blocked with a blocking agent to avoid non-specific binding. In an embodiment, the blocking is bovine serum albumin, milk or milk proteins. In an embodiment, the blocking agent is bovine serum albumin (BSA), optionally at a concentration of 0.05 to 1%.

IV. Methods of Detection and Kits of the Disclosure

Also provided herein is a method for detecting the presence of C. difficile glutamate dehydrogenase in a test sample, comprising contacting said sample with an aptamer disclosed herein, an aptamer probe disclosed herein or a biosensor system disclosed herein under conditions for a binding-induced conformational change in the aptamer to occur, and detecting a signal, wherein the aptamer, the aptamer probe or the biosensor system comprises a circular DNA aptamer, wherein the circular DNA aptamer comprises a sequence of SEQ ID NO: 6, 7, 14, or 16, or a functional fragment or modified derivative thereof, and wherein detection of a signal indicates the presence of C. difficile GDH in the test sample and lack of signal indicates that C. difficile GDH is not present. In an embodiment, the circular DNA aptamer comprises a sequence of SEQ ID NO: 6 or 14, or a functional fragment or modified derivative thereof. In an embodiment, the circular DNA aptamer comprises a sequence of SEQ ID NO: 7 or 16, or a functional fragment or modified derivative thereof. In an embodiment, the circular DNA aptamer comprises a circular DNA aptamer comprising a sequence of SEQ ID NO: 6, or a functional fragment or modified derivative thereof. In an embodiment, the circular DNA aptamer comprises a circular DNA aptamer comprising a sequence of SEQ ID NO: 7, or a functional fragment or modified derivative thereof. In an embodiment, the circular DNA aptamer comprises a circular DNA aptamer comprising a sequence of SEQ ID NO: 14, or a functional fragment or modified derivative thereof. In an embodiment, the circular DNA aptamer comprises a circular DNA aptamer comprising a sequence of SEQ ID NO: 16, or a functional fragment or modified derivative thereof.

In an embodiment, the test sample is a biological sample from a subject suspected of having a C. difficile infection. In one embodiment, the biological sample is a sample of excrement, for example, stool, from the subject.

In another aspect, the disclosure provides a method for detecting the presence of a target C. difficile glutamate dehydrogenase in a test sample, comprising:
a) contacting the test sample with a circular DNA aptamer to form a mixture, wherein the circular DNA aptamer is capable of binding to the target C. difficile glutamate dehydrogenase to form a complex;
b) separating the complex from the mixture; and c) detecting a signal from the complex through rolling circle amplification (RCA) of the circular DNA aptamer,
wherein detection of a signal indicates the presence of the target molecule in the test sample and the lack of signal indicates the absence of the target molecule, and
wherein the circular aptamer comprises a circular DNA aptamer comprising the sequence of SEQ ID NO: 6, 7, 14, or 16, or a functional fragment or modified derivative thereof.

In an embodiment, the circular DNA aptamer comprises a circular DNA aptamer comprising a sequence of SEQ ID NO: 6, or a functional fragment or modified derivative thereof. In an embodiment, the circular DNA aptamer comprises a circular DNA aptamer comprising a sequence of SEQ ID NO: 7, or a functional fragment or modified derivative thereof. In an embodiment, the circular DNA aptamer comprises a circular DNA aptamer comprising a sequence of SEQ ID NO: 14, or a functional fragment or modified derivative thereof. In an embodiment, the circular DNA aptamer comprises a circular DNA aptamer comprising a sequence of SEQ ID NO: 16, or a functional fragment or modified derivative thereof.

In an embodiment, the circular DNA aptamer is a circular DNA aptamer probe. In an embodiment, the circular DNA aptamer is attached directly or indirectly to a solid support. In an embodiment, the circular DNA aptamer indirectly via GDH attached to the solid support. In an embodiment, the method comprises separating the complex from the solid support. In an embodiment, the solid support comprises nitrocellulose filter disc or magnetic beads. In an embodiment, the solid support comprises magnetic beads. In an embodiment, the solid support comprises magnetic beads coated with a metal. In an embodiment, the nitrocellulose filter disc comprises a pore size 0.45 or 0.22 um. In an embodiment, the solid support comprises magnetic beads coated with a metal. In an embodiment, the solid support comprises magnetic beads comprising silica, polystyrene, or agarose, coated with nickel, cobalt, copper, iron, zinc, or aluminum. In an embodiment, the magnetic beads comprises silica, polystyrene, or agarose. In an embodiment, the metal comprises nickel, cobalt, copper, iron, zinc, or aluminum. In an embodiment, the solid support comprises Nickel

Nitrilotriacetic acid magnetic beads (NiMBs). In an embodiment, the denaturing comprises heating or urea treatment, optionally 10M urea. In an embodiment, the aptamer probe comprises an aptamer and a detectable label. In an embodiment, the test sample is a blood sample, a urine sample, a saliva sample, or a stool sample. In an embodiment, the test sample is a stool sample.

The disclosure provides herein a method of RCA comprising:
annealing a primer to a circular template;
wherein the circular template comprises a region complementary to the primer; and
amplifying the circular template under conditions that allow rolling circle amplification,
wherein the circular template comprises a circular DNA aptamer comprising a sequence of SEQ ID NO: 6, 7, 14, or 16, or a functional fragment or modified derivative thereof.

In an embodiment, the circular DNA aptamer comprises a circular DNA aptamer comprising a sequence of SEQ ID NO: 6, or a functional fragment or modified derivative thereof. In an embodiment, the circular DNA aptamer comprises a circular DNA aptamer comprising a sequence of SEQ ID NO: 7, or a functional fragment or modified derivative thereof. In an embodiment, the circular DNA aptamer comprises a circular DNA aptamer comprising a sequence of SEQ ID NO: 14, or a functional fragment or modified derivative thereof. In an embodiment, the circular DNA aptamer comprises a circular DNA aptamer comprising a sequence of SEQ ID NO: 16, or a functional fragment or modified derivative thereof.

In an embodiment, the method of RCA further comprises subjecting the product of the amplification to restriction digestion. In an embodiment, upon restriction enzyme digestion of the product, the concatemer is separated into individual monomer amplicons, which can then optionally be detected or quantified. A person skilled in the art would understand that any restriction recognition sites would be compatible.

In another embodiment, the method of RCA further comprises c) detecting the product of the RCA. Methods for detection of products of RCA are known in the art. For example, a nucleotide probe that is complementary to a portion of the concatemer can be labeled and incubated with the product under stringent conditions to allow for hybridization and subsequent detection of the label. Detection includes qualitative and quantitative detection.

In another aspect, the disclosure provides a method for detecting the presence of a target C. difficile glutamate dehydrogenase in a test sample, comprising:
contacting the test sample with a synthetic target C. difficile glutamate dehydrogenase-coated solid support coupled with a circular DNA aptamer to form a complex,
wherein the circular DNA aptamer is capable of binding to native form of the target C. difficile glutamate dehydrogenase;
wherein the circular DNA aptamer comprises a circular DNA aptamer comprising a sequence of SEQ ID NO: 6, 7, 14, or 16, or a functional fragment or modified derivative thereof; and
wherein the circular DNA aptamer has lower or equivalent affinity for the synthetic target C. difficile glutamate dehydrogenase compared to the native form of the target C. difficile glutamate dehydrogenase present in a test sample.

In an embodiment, the circular DNA aptamer comprises a circular DNA aptamer comprising a sequence of SEQ ID NO: 6, or a functional fragment or modified derivative thereof. In an embodiment, the circular DNA aptamer comprises a circular DNA aptamer comprising a sequence of SEQ ID NO: 7, or a functional fragment or modified derivative thereof. In an embodiment, the circular DNA aptamer comprises a circular DNA aptamer comprising a sequence of SEQ ID NO: 14, or a functional fragment or modified derivative thereof. In an embodiment, the circular DNA aptamer comprises a circular DNA aptamer comprising a sequence of SEQ ID NO: 16, or a functional fragment or modified derivative thereof.

The phrase “contacting the sample” or “contacting the test sample”, or a derivative thereof refers to incubating the sample, which has been processed, with the aptamer or aptamer probe, which allows any GDH in the sample to bind to the aptamer and induce a conformational change in the aptamer or aptamer probe. In the biosensor system comprising an aptamer or an aptamer probe disclosed herein, the aptamer-GDH or aptamer probe-GDH is then desorbed from the nanomaterial and the fluorescent signal can be detected.

Detection of the signal can be performed using any available method, including, for example, colorimetric, electrochemical and/or spectroscopic methods, depending on the label on the aptamer. The detection can simply be detection of the direct product formed, for example, by reaction or interaction of the aptamer with GDH, if the product being formed possesses a color (or any signal, such as a fluorescent signal) that is intense enough to be detected and that is distinct from the color (or signal) of any of the starting reagents. In some embodiments, detection of the signal comprises rolling circle amplification (RCA) of a circular aptamer. In some embodiments, the detection means is not a separate component of the biosensor system, but is instead formed during the assay and therefore is an inherent part of the biosensor system. In a further embodiment, the detection means comprises a separate entity that reacts or interacts with the direct product formed by reaction of, for example, the aptamer and the GDH, the reaction with the separate entity resulting in a distinct detectable signal.

The initial GDH screening test can be used in clinical diagnosis of CDI. Accordingly, further provided herein is a method of detecting C. difficile infection in a subject comprising testing a sample from the subject for the presence of C. difficile GDH by the method disclosed herein; and if GDH is present, further comprising testing the sample for the presence of C. difficile toxins A and B (with a protein sequence as set out in Genbank Accession No. CAA63564.1 (SEQ ID NO: 19) and CAA63562.1 (SEQ ID NO: 20), respectively); wherein the presence of GDH and the presence of toxins indicates that the subject has a C. difficile infection. In an embodiment, testing for the presence of C. difficile toxins comprises a cell cytotoxicity neutralization assay, a toxin enzyme immunoassay or detection of toxin genes using PCR.

In another embodiment, the method further comprises treating the subject for C. difficile infection if GDH and toxins are present. Also provided is use of a medicament to treat a subject that has been identified as having a C. difficile infection by the method disclosed herein. In an embodiment, treatment of C. difficile or a medicament for treatment of C. difficile comprises antibiotics such as metronidazole, vancomycin or fidaxomicin.

Even further provided herein is a kit for detecting C. difficile glutamate dehydrogenase, wherein the kit comprises an aptamer disclosed herein, an aptamer probe disclosed herein and/or a biosensor system disclosed herein and instructions for use of the kit. In an embodiment, the kit further comprises components of RCA described herein. In an embodiment, the kit further comprise a synthetic GDH. In an embodiment, the kit further comprises a blocking agent for non-specific binding to a solid support, such as bovine serum albumin (BSA), milk or milk proteins. In an embodiment, the blocking agent is BSA, optionally at a concentration of 0.05 to 1%.

### EXAMPLES

The following non-limiting examples are illustrative of the present disclosure:

### Example 1

### Selection of Captamer for Glutamate Dehydrogenase (GDH) From Clostridium difficile.

### Experiment Section

### Example 1A

### Oligonucleotides and Other Materials

All DNA oligonucleotides (Table 2) were obtained from Integrated DNA Technologies (IDT), and purified by standard 10% denaturing (8 M urea) polyacrylamide gel electrophoresis (dPAGE). T4 DNA ligase, phi29 DNA polymerase, T4 polynucleotide kinase (PNK) and deoxyribonucleoside 5′-triphosphates (dNTPs) were purchased from Thermo Scientific (Ottawa, ON, Canada). Thermus thermophilus DNA polymerase was obtained from Biotools. Recombinant (his-tagged) glutamate dehydrogenase (rGDH, isoelectric point of 5.60 and molecular weight of 46,000 Da; expressed and purified from E. coli cells), native GDH (nGDH), TcdA and TcdB were obtained from Pro-Lab Diagnostics (Toronto, ON, Canada). Ni-NTA magnetic agarose beads were obtained from QIAGEN. [α-³²P]deoxy-GTP and γ[³²P]-ATP were acquired from Perkin Elmer (Woodbridge, ON, Canada). All other chemicals were purchased from Sigma-Aldrich (Oakville, ON, Canada) and used without further purification.

### Example 1B

### Instruments

The autoradiogram and fluorescent images of gels were obtained using a Typhoon 9200 variable mode imager (GE Healthcare) and analyzed using Image Quant software (Molecular Dynamics). Time-dependent fluorescence emission measurements were performed using a BioRad CFX96 qPCR system and Genie II portable fluorimeter platform.

### Example 1C

### In Vitro Selection of Captamers

Library preparation. The DNA library DL1 (SEQ ID NO: 1) was chemically synthesized and purified by 10% dPAGE. Purified DL1 was replicated using a previously described protocol [10]. Briefly, 1 nM of purified DL1 was amplified by PCR for 5 cycles with a biotinylated primer (˜32-fold amplification). Following the amplification, single-stranded DNA was prepared using streptavidin modified agarose beads and elution with an alkaline solution. ⅓^{rd} of the thus prepared DNA pool (˜15 nmoles) was used for preparing the required circular DNA library (cirDNA) through template-assisted ligation as previously described [14]. Briefly, DL1 was first phosphorylated as follows: a reaction mixture (100 μL) was made to contain 3 μM linear oligonucleotide, 10 U PNK (U: unit), 1× PNK buffer A (50 mM Tris-HCl, pH 7.6 at 25° C., 10 mM MgCl₂, 5 mM DTT, 0.1 mM spermidine), and 1 mM ATP. The mixture was incubated at 37° C. for 1 h, followed by heating at 90° C. for 10 min. Equimolar DNA ligation template (LT1 (SEQ ID NO: 5)) was then added, heated at 90° C. for 60 s and cooled to room temperature (RT) for 20 min. Then, 15 μL of 10×T4 DNA ligase buffer (400 mM Tris-HCl, 100 mM MgCl₁₂, 100 mM DTT, 5 mM ATP, pH 7.8 at 25° C.) and 20 U of T4 DNA ligase were added (total volume 150 μL). This mixture was incubated at RT for 2 h before heating at 90° C. for 10 min to deactivate the ligase. The ligated cDNAs were concentrated by standard ethanol precipitation and purified by 10% dPAGE.

The SELEX procedure was performed according to reported method with modifications [9a]. In the first round, the cirDNA library (10 nmol), which was denatured at 90° C. for 10 min and then cooled on ice for 15 min, was incubated with bead-bound rGDH (138 pmol) with rotation for 30 min at room temperature in 500 μL of 1× binding buffer (pH 7.2) containing 50 mM HEPES, 150 mM NaCl, 10 mM MgCl₂, 5 mM KCl and 0.02% Tween-20. The tube was then applied to a magnet (Qiagen), the supernatant was removed, and the beads were washed three times with 1× binding buffer (500 μL). rGDH and bound aptamers were eluted with 300 μL of elution buffer (1× binding buffer containing 500 mM imidazole) at 90° C. for 15 min with gentle shaking. After recovery by ethanol precipitation, the cirDNA was amplified by standard PCR with FP1 (SEQ ID NO: 2) and RP1 (SEQ ID NO: 3). Thermal cycles were typically performed as follows: 94° C. for 30 s; 16 cycles of 94° C. for 30 s, 50° C. for 45 s and 72° C. for 40 s; 72° C. for 5 min. A small aliquote of the PCR product (1%) was taken as an input in PCR2 with the use of FP1 (SEQ ID NO: 2) and RP2 (SEQ ID NO: 4; which contained internal C3 spacer) under the same reaction conditions. The purpose of the second PCR was to make the nonaptamer-coding DNA strand 12 nucleotides longer than the coding strand. The amplified DNA was then separated and purified by 10% dPAGE. The concentration of rGDH was kept at 138 pmol for rounds 1-6 and then reduced to 46 pmol for rounds 7-12. The DNA pool from round 12 was used for deep sequencing using an Illumina Miseq system at the Farncombe Metagenomics Facility, McMaster University (Hamilton, ON, Canada).

### Example 1D

### Pull-Down Assay

100 μL of 1× binding buffer containing 100 nM γ-[³²P]-labeled captamer candidates was first denatured at 90° C. for 10 min, followed by incubation at RT for 20 min. The pull-down assays were conducted in a total volume of 300 μL of 1× binding buffer containing 50 μL of bead-bound rGDH (final concentration ˜50 nM) and 100 μL of 100 nM captamer candidates for 30 min with gentle rotation. In parallel, negative controls consisting of the captamers incubated with uncoated beads were included. Following three washing steps, proteins and bound aptamers were eluted with 30 μL of elution buffer (20 mM Tris-HCl, 500 mM imidazole, pH 7.5) by shaking at 90° C. for 10 min. The resultant mixtures were then analyzed by 10% dPAGE, and quantified by Image Quant software. For determination of the dissociation constants (K_{d}), 100 μL of 2 μM γ-[³²P]-labeled aptamers was first denatured in 1× binding buffer at 90° C. for 10 min, followed by incubation at RT for 20 min. Pull-down assays were conducted in a total volume of 300 μL of 1× binding buffer containing 10 μL of bead-bound rGDH (final concentration ˜9.2 nM) and 100 μL of aptamers with serial dilutions for 30 min with gentle rotation. Following washing steps, the bound DNA was then analyzed by 10% dPAGE. The apparent dissociation constants (K_{d}) of each aptamer candidate were obtained by fitting the fractional bound DNA to the total aptamer concentration using a one-site binding model.

### Example 1E

### Dot Blotting Assay

A total of 2 μL of rGDH (2.3 μg/uL) was spotted onto a nitrocellulose membrane (Millipore HF180) and was allowed to dry at RT for 10 min. Then, the membrane was blocked with 10 μL of 1% BSA in 1× binding buffer for 20 min. After washed once with 20 μL of 1× binding buffer, 10 μL of 100 nM γ-[³²p]-labeled Apt-2 or Apt-4 were added and incubated for 20 min. The paper plate was placed in a trough containing 2 mL of washing buffer for 5 min, nitrogen-dried, and exposed to a phosphor-imaging screen.

### Example 1F

### Filter Binding Assay

Serially diluted nGDH were first incubated with 10 nM γ[³²P]-labeled Capt-4 in 20 μL of 1× binding buffer containing 1% BSA for 30 min. The mixtures were then spotted onto wax-printed 96-microzone paper plates (Whatman® Grade 1 chromatography paper), which will pass through the filter by capillary force. After washing with 1× binding buffer three times (20 μL each time), the paper was dried at RT and exposed to a phosphor-imaging screen before scanning.

### Example 1G

### Competitive Binding Assay

300 μL of 1× binding buffer containing 200 nM γ-[³²P]-labeled Lapt-2 and Capt-2 or Capt-4, which had been denatured at 90° C. for 5 min and then cooled at RT, was incubated with 10 μL of bead-bound rGDH (final concentration ˜9.2 nM) with rotation for 30 min. The tube was then applied to a magnet (Qiagen), the supernatant was removed, and the beads were washed three times with 500 μL of 1× binding buffer. The bound DNA was then analyzed by 10% dPAGE. For the controls, a random DNA sequence (RDS) was used instead of Lapt-2.

### Example 1H

### Sandwich Assay

To facilitate the immobilization of Lapt-2 on paper, the inventors first prepared the streptavidin-biotinylated Lapt-2 conjugate according to reported method [5f]. A desired volume (˜5 μL) of the above solution was then printed onto the nitrocellulose membrane (Millipore HF180) with a Scienion SciFlex Arrayer 10 Non-Contact Microarray Printer and allowed to dry at RT. The plates were then blocked with 20 μL of 1× binding buffer containing 1% BSA. After incubating at RT for 10 min and washing once by 20 μL of 1× binding buffer, 10 μL of 100 nM rGDH was added and allowed to bind for 20 min. Then 10 μL of 200 nM γ[³²P]-labeled Capt-4 was added and incubated for another 20 min. The paper plate was placed in a trough containing 2 mL of washing buffer for 5 min, nitrogen-dried, and exposed to a phosphor-imaging screen.

### Example 1I

### Target Detection

300 μL of 1× binding buffer containing 300 nM Capt-4, which had been denatured at 90° C. for 2 min and then cooled at RT, was incubated with 10 μL of bead-bound rGDH (final concentration ˜20 nM) with rotation for 15 min. 10 μL of 1% BSA was added and incubated for 10 min. The supernatant was removed, and the beads were washed three times before being re-dispersed in 40 μL of 1× binding buffer. Then 10 μL of nGDH with different dilutions in pure buffer or unformed faecal specimens was added. Following 15 min incubation with gentle shaking, the supernatant was transferred to a new tube and heated at 90° C. for 2 min. To this mixture were added 10 μL of 10× RCA reaction buffer (330 mM Tris acetate, 100 mM magnesium acetate, 660 mM potassium acetate, 1% (v/v) Tween-20, 10 mM DTT, pH 7.9), 5 U of phi29 DNA polymerase, 2 μL of dNTPs (10 mM) and 1 μL of DPI (100 μM), and the resultant mixture (final volume: 100 μL) was incubated at 30° C. for 30 min before being analyzed by 0.6% agarose gel electrophoresis. A similar experiment was carried out using non-target proteins (BSA, TcdA and TcdB from C. difficile) to evaluate the specificity. For the real-time monitoring of RCA at various nGDH concentrations, these reactions were monitored using a BioRad CFX96 qPCR system or Genie II portable fluorimeter platform set to a constant temperature of 30° C., and the fluorescence intensity was recorded.

### Example 1J

### Faecal Sample Analysis

50 μL of unformed faeces were first transferred to 100 μL of Sample Diluent (Pro-Lab Diagnostics). The sample was vortexed for 30 s to thoroughly emulsify the specimen. This was followed by the addition of the above prepared bead-bound rGDH/Capt-4 mixture in 200 μL of 1× binding buffer. Following 15 min incubation with gentle shaking, the supernatant was transferred to a new tube and heated at 90° C. for 5 min. To 50 μL of this mixture were added 10 μL of 10×RCA reaction buffer (330 mM Tris acetate, 100 mM magnesium acetate, 660 mM potassium acetate, 1% (v/v) Tween-20, 10 mM DTT, pH 7.9), 5 U of phi29 DNA polymerase, 2 μL of dNTPs (10 mM) and 1 μL of LT1 (SEQ ID NO: 5; 100 μM). These reactions (final volume: 100 μL) were monitored using a Genie II portable fluorimeter platform set to a constant temperature of 30° C., and the fluorescence intensity was recorded.

### Example 2

### Advantages of Performing Aptamer Selection With a Circular DNA Library

To demonstrate the aforementioned advantages, the inventors carried out captamer selection targeting glutamate dehydrogenase (GDH) from Clostridium difficile (C. difficile), a proven antigen for diagnosing C. difficile infections (CDI) that cause many annual global outbreaks [7]. GDH is an excellent biomarker for diagnosing CDI because it is at fairly high levels (˜0.02-20 nM) in the feces of CDI patients but undetectable in the stool of healthy people [8]. In addition, because the inventors have shown a linear DNA aptamer for the same GDH [9], head-to-head comparisons of the binding to the same target by linear and circular aptamers can be conducted. For this consideration, the inventors conducted the aptamer selection with the same library used for the linear aptamer selection, which was replicated using a published protocol (see Example 1C and ref [10]).

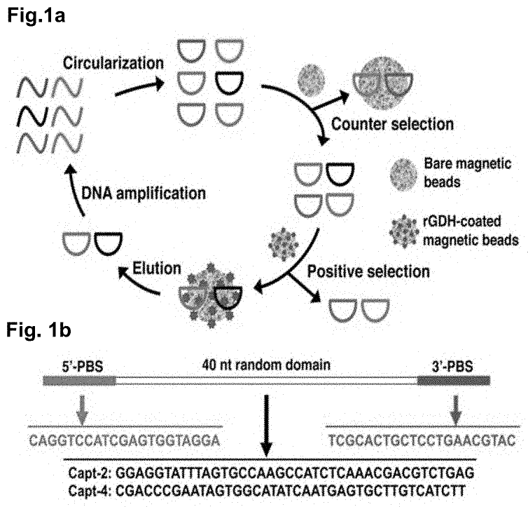

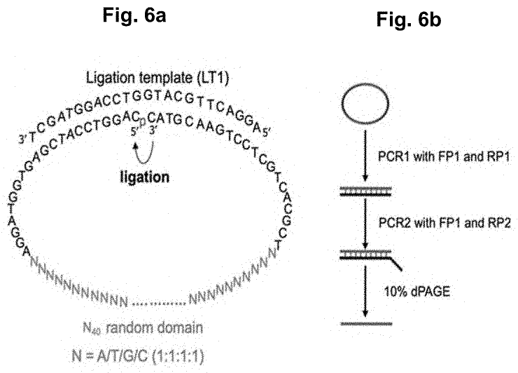

The selection strategy included 5 key steps, as illustrated in FIG. la. The first step was the preparation of the DNA library by the end-to-end ligation (circularization) of the linear DNA pool, DL1 (SEQ ID NO: 1; which contained a 40-nucleotide random domain; see FIG. 1b for its sequence). This step used a DNA oligonucleotide, named LT1 (SEQ ID NO: 5; see Table 2 for the sequences of all the DNA oligonucleotides used in this work), that binds the 5′-end and 3′-end of DL1 to create a duplex structure for T4 DNA ligase-mediated ligation (FIG. 6a). The circular DNA pool was then applied to the bare Ni-NTA magnetic beads (NiMBs) in a counter-selection step to remove bead-binding DNA molecules. The unbound DNA sequences were incubated with the NiMBs coated with histidine-tagged recombinant GDH (rGDH). The bound DNA molecules were eluted by heating, and then amplified by PCR. This step used a regular forward DNA primer and a modified reverse primer containing a non-amplifiable A12 extension at its 5′-end, which made the aptamer-containing sense DNA strand shorter than the antisense strand (see FIG. 6b for complete information regarding this step). After purification using denaturing (7 M urea) gel electrophoresis (dPAGE) [9a] and circularization, the amplified sense DNA strand was used for the next round of selection.

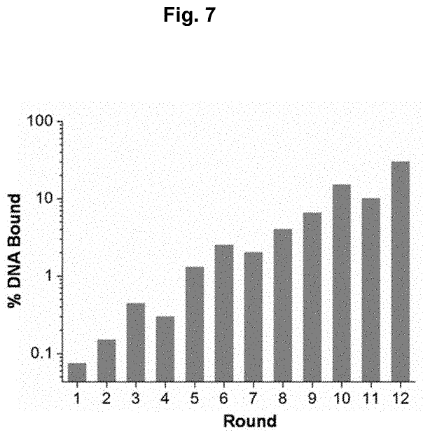

˜10¹⁵ distinct circular DNA molecules were used as the initial DNA library. 12 rounds of selection were carried out, and the fraction of the DNA bound to rGDH-NiMBs increased in each round (FIG. 7). The enriched sequences from round-12 were subjected to high-throughput DNA sequencing and the results are provided in Table 3. 321,489 sequence reads were obtained; however, the top 5 sequences, named Capt-1 to Capt-5, account for ˜46% of them, indicating the selection method was effective in sequence enrichment.

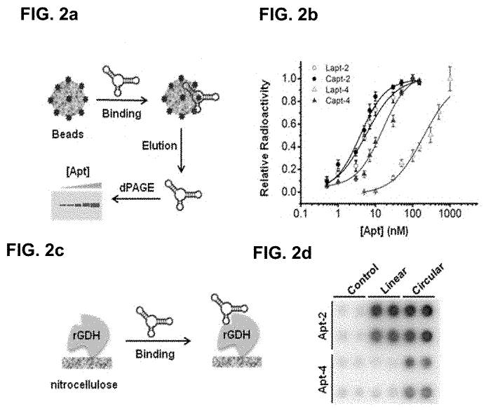

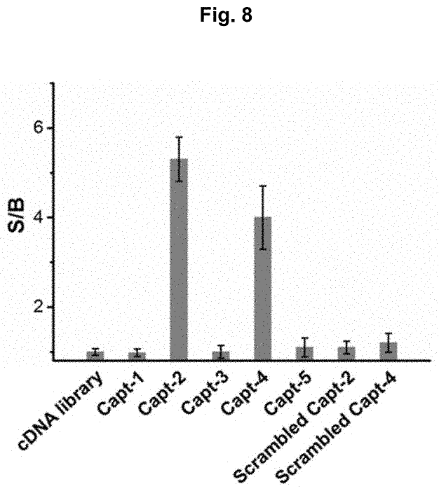

Interestingly, the sequences of Capt-1, 2, 3 and 5 were also observed in the linear GDH-binding aptamer selection (which were named Anti-G6, Anti-G1, Anti-G5, Anti-G4 respectively) [9a], an unsurprising finding given that the same replicated DNA library was used for both. However, Capt-4 was a new sequence that was not observed in the previous selection. A pull-down assay was then carried out for radiolabeled versions of the top 5 sequences. In this assay, each captamer candidate was incubated with bare NiMBs or rGDH-coated NiMBs, followed by elution of the bound captamer and radioactivity analysis by dPAGE (FIG. 2a). While Capt-1, Capt-3 and Capt-5 exhibited strong binding to bare NiMBs and minimal binding to rGDH (similar to inventors' previous observations), Capt-2 (SEQ ID NO: 6) and Capt-4 (SEQ ID NO: 7) showed strong binding to rGDH-coated NiMBs and minimal binding to beads alone (FIG. 8). Scrambled Capt-2 (SEQ ID NO: 11) and Capt-4 (SEQ ID NO: 12) sequences showed no affinity to rGDH (FIG. 8), indicating that the recognition by the two aptamers is sequence-specific.

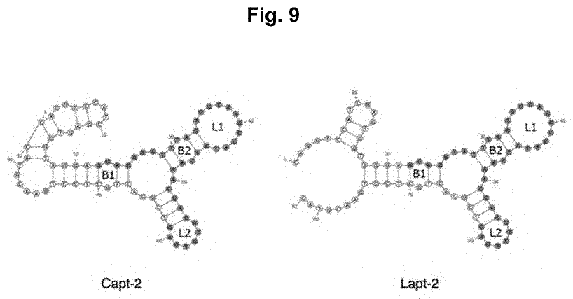

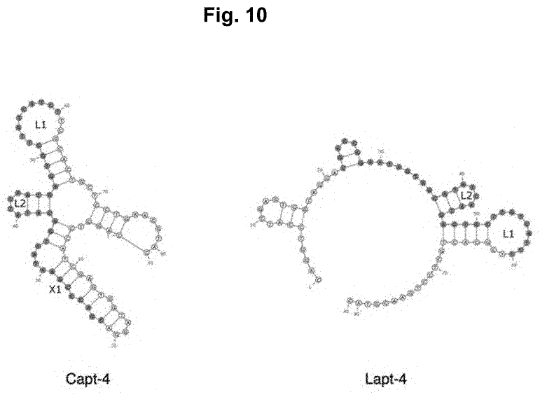

The inventors measured the dissociation constant (K_{d}) for Capt-2 and Capt-4 using the same pull-down assay, along with their linear counterparts, Lapt-2 and Lapt-4 (FIG. 2b). Capt-2 and Lapt-2 exhibited similar K_{d} values (5.6±2.1 nM and 4.4±2.5 nM, respectively), consistent with the observation that the same aptamer was seen in both selections. The structural prediction with the m-fold program (http: //unafold.rna.albany.edu/?q=mfold) also provides a good rationale for the functionality of aptamer 2 in both circular and linear configurations, as the random-sequence nucleotides are predicted to form the same structural elements in both forms (FIG. 9). However, while Capt-4 showed excellent affinity for rGDH with a K_{d} of 12.3±4.6 nM, Lapt-4 showed much reduced affinity, with a K_{d} of 293±15 nM, representing ˜25-fold affinity reduction (Table 1). A comparison of the predicted secondary structures for Lapt-4 and Capt-4 reveals significant disparities (FIG. 10). Their difference in binding affinity is also consistent with the lack of observation of aptamer 4 in the linear library-based selection, as it is clear that this particular aptamer requires the circular configuration to achieve a high level of binding to rGDH.

The striking difference seen with aptamers 2 and 4 was also evaluated via dot blotting assays with rGDH being immobilized onto nitrocellulose membranes (FIG. 2c). Equivalent levels of binding to rGDH were observed with both Lapt-2 and Capt-2, while only the circular form of aptamer 4 (Capt-4) produced detectable binding to rGDH (FIG. 2d).

Taken together, the results above highlight the added advantage of performing aptamer selection with a circular DNA library, as this can result in the discovery of aptamers that cannot be obtained from a linear library.

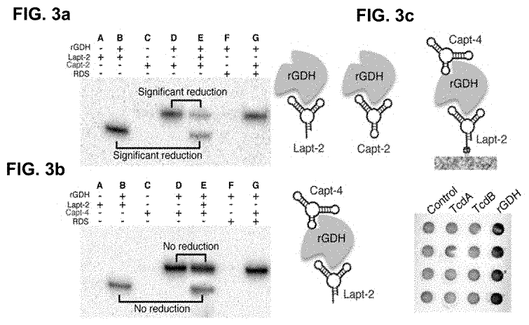

Interestingly, Capt-2 and Capt-4 recognize distinct epitopes on rGDH, a conclusion drawn from both a competitive binding assay (FIG. 3a and FIG. 3b) and a sandwich dot blotting assay (FIG. 3c). FIG. 3a was designed to show that Capt-2 and Lapt-2 competed with each other for binding to rGDH using the pull-down assay. Lanes B and D show that the amount of radioactive Lapt-2 and radioactive Capt-2 pulled down by rGDH-coated NiMBs when each radioactive aptamer was individually incubated with the beads, while Lane E depicts the amount of Lapt-2 and Capt-2 pulled down when they were combined and incubated with the beads. The significant reduction of both Lapt-2 and Capt-2 in Lane E is consistent with the expected competitive binding between Lapt-2 and Capt-2. FIG. 3b was set up identically except that radioactive Capt-4 was used to substitute Capt-2. However, the outcome was drastically different: the amount of Lapt-2 and Capt-4 pulled down from their mixtures was equivalent to the amount pulled down from the single aptamer samples, without wishing to be bound by theory, suggesting that these aptamers bound different epitopes on rGDH. As a control, a random DNA sequence (RDS) with no binding activity (Lane F) was used to replace Lapt-2 for each competition assay. It had no impact on the amount of Capt-2 or Capt-4 pulled down when it was combined with each circular aptamer (Lane G).

That aptamers 2 and 4 recognize different epitopes was further confirmed using a dot blotting sandwich assay in which biotinylated Lapt-2 was immobilized on streptavidin-coated paper plates to capture rGDH and ³²P-labeled Capt-4 was used to report captured rGDH on the paper. As shown in FIG. 3c, a strong signal was observed in the presence of rGDH, but not in the presence of C. difficile TcdA and TcdB (controls).

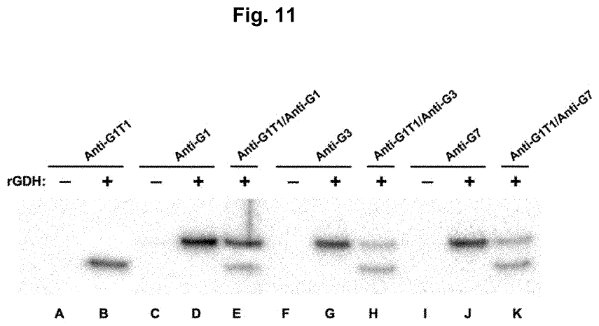

Inventors' selection with the linear DNA library resulted in 3 linear aptamers: Anti-G1 (which is Capt-2), Anti-G3 and Anti-G7 [9a] (see Table 2 for their sequences). The competition assay illustrated in FIG. 11 clearly indicates that all 3 linear aptamers recognize the same epitope. This finding together with the observation that Capt-4 binds rGDH at a different site points to an additional advantage of performing selection with a circular DNA library: it generates aptamers with unique binding properties owing to their circular topology.

The inventors next applied circular aptamers for the diagnosis of CDI.

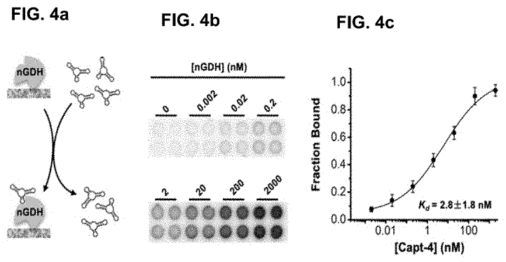

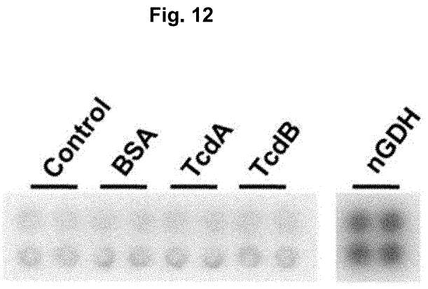

Achieving this objective requires that the circular aptamers recognize native GDH (nGDH) from C. difficile. The inventors confirmed the binding of Capt-4 to nGDH using the filter binding assay (FIG. 4a and FIG. 4b). The inventors also measured the K_{d} of Capt-4 for nGDH using the same assay (FIG. 4c), which was 2.8±1.8 nM, ˜4-fold higher than its affinity for rGDH, presumably due to the hexameric structure of nGDH [11]. Three non-target proteins, BSA and C. difficile toxins TcdA and TcdB [12], were also tested. No binding was observed to the non-target proteins, indicating that Capt-4 is highly specific for GDH (FIG. 12).

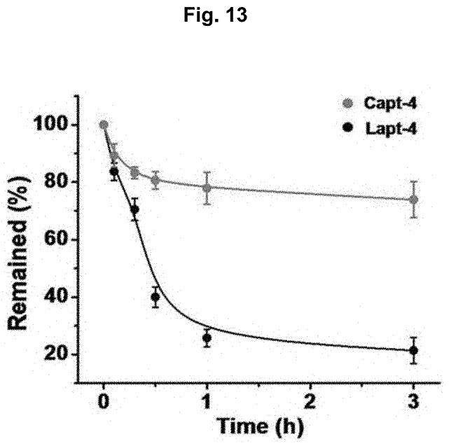

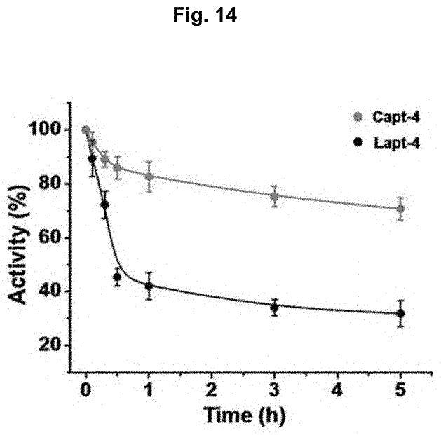

Human faeces are commonly used specimens in diagnosing CDI [13], and therefore, it was next examined the molecular integrity of Capt-4 and Lapt-4 in such samples. The half-life of Capt-4 was >3 h, compared to 0.4 h observed for Lapt-4 (FIG. 13). The measured functional half-lives of Lapt-4 and Capt-4 were 0.35 h and >5 h respectively (FIG. 14), in good agreement with their nucleolytic stability. These results indicated that captamers were more desirable than linear aptamers for diagnostic applications using faecal samples.

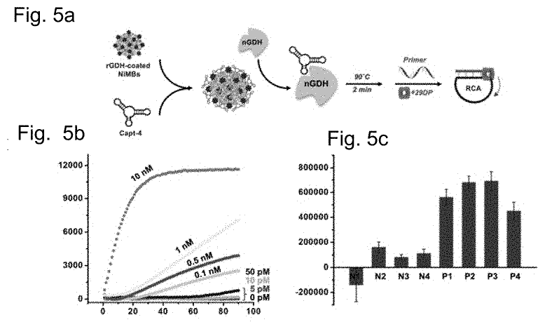

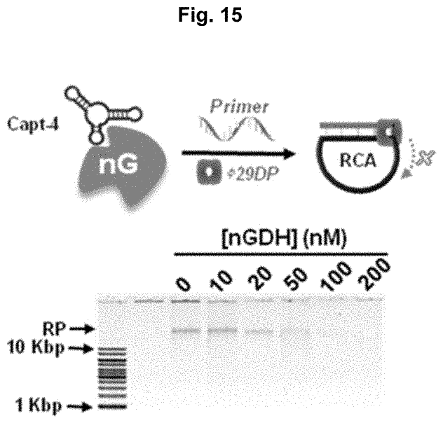

The inventors next designed a biosensing assay for the detection of nGDH from human faeces that takes advantage of the observation that Capt-4 shows significantly better affinity to nGDH (K_{d}=2.8 nM) over rGDH (K_{d}=12.3 nM). The strategy features rGDH-coated NiMBs pre-bound with Capt-4; the presence of nGDH from a test sample causes the release of Capt-4, which is then used for RCA to produce long-chain DNA molecules with repetitive sequences (FIG. 5a) [5]. The use of RCA converts the detection of the target to the detection of DNA amplicons, offering significantly better detection sensitivity [5d-g]. Importantly, the captamer acts both as the molecular recognition element and as the circular DNA template for RCA. A heat-denaturation step (90° C. for 2 min) was included following magnetic separation, to liberate Capt-4 from the nGDH-Capt-4 complex for RCA, as the inventors found that nGDH binding reduced Capt-4's RCA efficiency, presumably due to its strong affinity to nGDH (FIG. 15).

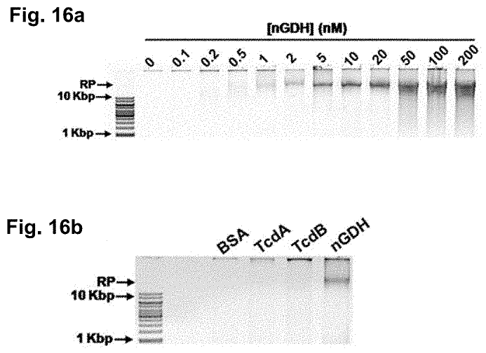

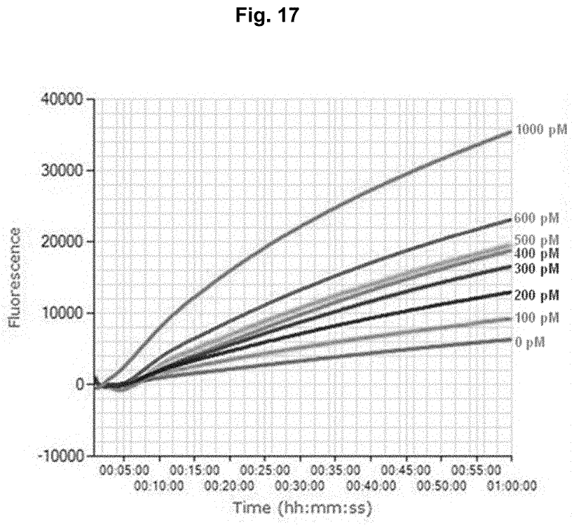

The inventors analyzed the production of RCA products in response to increasing concentrations of nGDH. The RCA products were observed by agarose gel analysis when the nGDH concentration was at 0.5 nM or higher (FIG. 16a). No signal was observed with BSA, TcdA and TcdB (FIG. 16b). The RCA reactions were also monitored in real time with SYBR Gold that binds RCA products to generate fluorescence. This approach was able to detect 10 μM nGDH at 90 min when using a conventional plate reader (FIG. 5b). A limit of detection of 100 μM in 60 min was also achieved by the portable Genie II fluorimeter (FIG. 17).

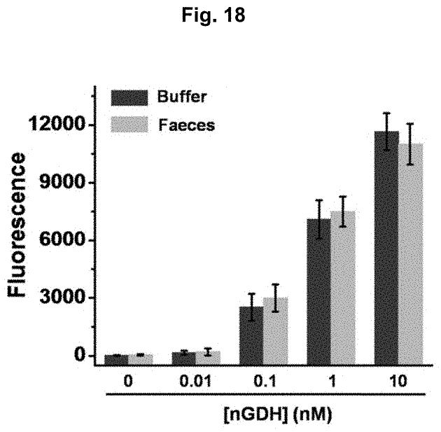

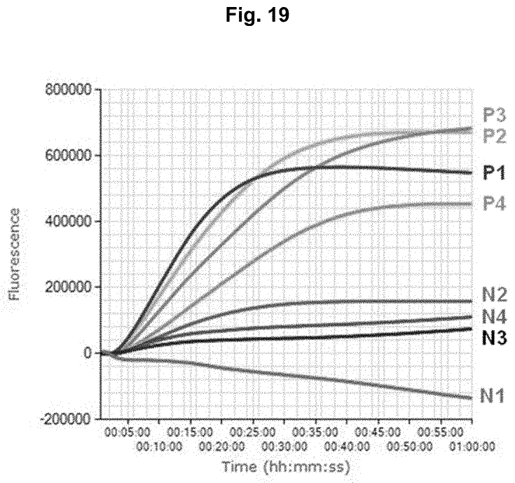

The inventors also performed the analysis of nGDH spiked into human faecal samples and found that the signal responses were comparable to the results obtained with pure buffer (FIG. 18). To investigate the applicability of the assay in clinical diagnosis of CDI, the inventors performed the assay on patient faecal samples. As shown in FIG. 5c and FIG. 19, signal intensities from CDI-positive patients were significantly higher than those of healthy controls.

### Example 3

### High-Affinity Circular DNA Aptamers That Specifically Recognize Glutamate Dehydrogenase (GDH) From C. difficile

The inventors undertook selection of protein-binding DNA aptamers from a circular DNA library, resulting in the isolation of two high-affinity circular DNA aptamers that specifically recognize glutamate dehydrogenase (GDH) from C. difficile, an established antigen for diagnosing C. difficile infections (CDI). One aptamer, Capt-2, has a sequence identical to an aptamer previously isolated from the same DNA library in a linear configuration and this aptamer works in both circular and linear forms. The other aptamer, Capt-4, which linear form sequence was not selected from the linear library and exhibits high affinity for GDH only in the circular form. Capt-4 and Capt-2 recognize different epitopes on GDH because they can simultaneously bind this protein.

Isolation of Capt-4 highlights the advantage of selecting aptamers from circular DNA libraries, without wishing to be bound by theory, because this approach not only can discover distinctive aptamers that do not exist in linear DNA libraries but can also produce high-affinity recognition elements with improved chemical and functional stabilities in biological media. Finally, the inventors show a sensitive diagnostic test for CDI, which takes advantage of three outstanding properties of Capt-4: high stability and functionality in human faeces, compatibility with rolling circle amplification, and higher binding affinity for native GDH from C. difficile relative to the recombinant version of GDH. This test is capable of detecting 10 μM native GDH in human faeces and can correctly identify patients with active CDI.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

TABLE 1
A comparison of K_{d} for different aptamer constructs.

| | Capt-2 | Lapt-2 | Capt-4 | Lapt-4 |
|---|---|---|---|---|
| K_{d} (nM)^{a} | 4.4 ± 2.5 | 5.6 ± 2.1 | 12.3 ± 4.6 | 293 ± 15 |

^{a}Pull-down assay was performed in 1× binding buffer (50 mM HEPES, 150 mM NaCl, 10 mM MgCl₂, 5 mM KCl and 0.02% Tween-20, pH 7.2) at 25° C.

TABLE 2
Sequences of DNA oligonucleotides used in this disclosure.

| Identifier | Name of DNA oligonucleotide | Sequence (5′-3′) |
|---|---|---|
| SEQ ID NO: 1 | DNA library (DL1) | CAGGT CCATC GAGTG GTAGG AN₄₀TCGC ACTGC |
| | | TCCTG AACGT AC |

Primers for PCR

| | | |
|---|---|---|
| SEQ ID NO: 2 | Forward primer (FP1) | CAGGT CCATC GAGTG GTAGG A |
| SEQ ID NO: 3 | Reverse primer (RP1) | GTACG TTCAG GAGCA GTGCG A |
| SEQ ID NO: 4 | Reverse primer (RP2) | AAAAA AAAAA AAXGT ACGTT CAGGA GCAGT |
| | | GCGA; X = internal C3 space |
| SEQ ID NO: 5 | Ligation template (LT1) | TCGAT GGACC TGGTA CGTTC AGGA |
| SEQ ID NO: 6 | Capt-2 | CAGGT CCATC GAGTG GTAGG AGGAG GTAT |
| | ( = Anti-G1 in Ref. 3)^{a} | TAGTG CCAAG CCATC TCAAA CGACG TCTGA |
| | | GTCGC ACTGC TCCTG AACGT AC |
| SEQ ID NO: 7 | Capt-4 | CAGGT CCATC GAGTG GTAGG ACGAC CCGAA |
| | | TAGTG GCATA TCAAT GAGTG CTTGT CATCT |
| | | TTCGC ACTGC TCCTG AACGT AC |
| SEQ ID NO: 8 | Anti-G3^{a} | CAGGT CCATC GAGTG GTAGG ATGCA GCGGA |
| | | CAGTG TGGGA CCATC GCTGC GGATG TATGA |
| | | ATCGC ACTGC TCCTG AACGT AC |
| SEQ ID NO: 9 | Anti-G7^{a} | CAGGT CCATC GAGTG GTAGG ACCCA ACCCA |
| | | CGATG CGCAA GAGGA ATGCA GCCTA CCAGC |
| | | ATCGC ACTGC TCCTG AACGT AC |
| SEQ ID NO: 10 | Anti-G1T1^{b} | TAGGA GGAGG TATTT AGTGC CAAGC CATCT |
| | | CAAAC GACGT CTGAG TCGCA CTGCT CCTG |

Scrambled Sequences^{c}

| | | |
|---|---|---|
| SEQ ID NO: 11 | Scrambled Capt-2 | CGGGA GTACG CATGT ATGAG ACACC CACGC |
| | | TATGT CCCTA CTATG AGGTG GAGAC TTGCT |
| | | ACGTG CATCG CAGAT CGGTC AA |
| SEQ ID NO: 12 | Scrambled Capt-4 | GAAGG ATTCG GACTA GGATC CGGTC CAACT |
| | | ACCGG TGCCC TAATG GCATC AAAAC TGCGT |
| | | ACCGA GCGTG TTTAT CTTTC TG |

^{a}Anti-G1, Anti-G3 and Anti-G7 were near aptamers previously selected from the linear library (Ref 3). Anti-G1 was the linear form of Capt-2 in this disclosure, i.e. Anti-G1 is Lapt-2.
^{b}Anti-G1T1 is a shortened version of Anti-G1 with full activity (Ref 3).
^{c}The scrambled sequences were generated using the following web based program: http://www.bioinformatics.org/sms2/shuffle_dna.html

TABLE 3
High-throughput sequencing results from selection round 12 pools

| | Random | | Multi- | % of |
|---|---|---|---|---|
| Identifier | Region ID | Sequence of random region(5′→3′) | plicity | Total |
| SEQ ID NO: 13 | RR-Capt- | CAGCT GTCGA CGCGT TACCG TGAAC | 99288 | 30.9 |
| | 1^{a} | GGAAC ACCGA TGACG | | |
| SEQ ID NO: 14 | RR-Capt- | GGAGG TATTT AGTGC CAAGC CATCT | 14151 | 4.4 |
| | 2^{a} | CAAAC GACGT CTGAG | | |
| SEQ ID NO: 15 | RR-Capt- | GTGTA GGGAC GCAAG ATGAA TGCAG | 13965 | 4.3 |
| | 3^{a} | CATAC CAGTC CCTAG | | |
| SEQ ID NO: 16 | RR-Capt- | CGACC CGAAT AGTGG CATAT CAATG | 11503 | 3.6 |
| | 4 | AGTGC TTGTC ATCTT | | |
| SEQ ID NO: 17 | RR-Capt- | TCAGT ACCAG TTCCC CAGGA GAATG | 10255 | 3.2 |
| | 5^{a} | CAGAT CCCCA GGTAC | | |

^{a}Capt-1,2,3,5 were also selected from the linear library (Ref. 3 above), which were given diffrent names: Capt-1 = AntiG6; Capt-2 = Anti-G1; Capt-3 = Anti-G5; Capt-5 = Anti-G4; RR: Random Region; Each captamer sequence also contains primer regions CAGGTCCATC GAGTGGTAGGA (SEQ ID NO: 2) or CAGGTCCATC GAGTGGTA (SEQ ID NO: 63) at the 5′ end and TCGCACTGCT CCTGAACGTA C (SEQ ID NO:64) at 3′ end flanking the random region.

TABLE 4
Sequence surrounding SEQ ID NO: 16

| | Ter- | Sequence surrounding |
|---|---|---|
| Identifier | minus | random region |
| SEQ ID NO: 21 | 5′ | CAGGT CCATC GAGTG GTAGG A |
| SEQ ID NO: 22 | 5′ | AGGTC CATCG AGTGG TAGGA |
| SEQ ID NO: 23 | 5′ | GGTCC ATCGA GTGGT AGGA |
| SEQ ID NO: 24 | 5′ | GTCCA TCGAG TGGTA GGA |
| SEQ ID NO: 25 | 5′ | TCCAT CGAGT GGTAG GA |
| SEQ ID NO: 26 | 5′ | CCATC GAGTG GTAGG A |
| SEQ ID NO: 27 | 5′ | CATCG AGTGG TAGGA |
| SEQ ID NO: 28 | 5′ | ATCGA GTGGT AGGA |
| SEQ ID NO: 29 | 5′ | TCGAG TGGTA GGA |
| SEQ ID NO: 30 | 5′ | CGAGT GGTAG GA |
| SEQ ID NO: 31 | 5′ | GAGTG GTAGG A |
| SEQ ID NO: 32 | 5′ | AGTGG TAGGA |
| SEQ ID NO: 33 | 5′ | GTGGT AGGA |
| SEQ ID NO: 34 | 5′ | TGGTA GGA |
| SEQ ID NO: 35 | 5′ | GGTAG GA |
| SEQ ID NO: 36 | 5′ | GTAGG A |
| SEQ ID NO: 37 | 5′ | TAGGA |
| SEQ ID NO: 38 | 5′ | AGGA |
| SEQ ID NO: 39 | 5′ | GGA |
| SEQ ID NO: 40 | 5′ | GA |
| SEQ ID NO: 41 | 5′ | A |
| SEQ ID NO: 42 | 3′ | TCGC ACTGC TCCTG AACGT AC |
| SEQ ID NO: 43 | 3′ | TCGC ACTGC TCCTG AACGT A |
| SEQ ID NO: 44 | 3′ | TCGC ACTGC TCCTG AACGT |
| SEQ ID NO: 45 | 3′ | TCGC ACTGC TCCTG AACG |
| SEQ ID NO: 46 | 3′ | TCGC ACTGC TCCTG AAC |
| SEQ ID NO: 47 | 3′ | TCGC ACTGC TCCTG AA |
| SEQ ID NO: 48 | 3′ | TCGC ACTGC TCCTG A |
| SEQ ID NO: 49 | 3′ | TCGC ACTGC TCCTG |
| SEQ ID NO: 50 | 3′ | TCGC ACTGC TCCT |
| SEQ ID NO: 51 | 3′ | TCGC ACTGC TCC |
| SEQ ID NO: 52 | 3′ | TCGC ACTGC TC |
| SEQ ID NO: 53 | 3′ | TCGC ACTGC T |
| SEQ ID NO: 54 | 3′ | TCGC ACTGC |
| SEQ ID NO: 55 | 3′ | TCGC ACTG |
| SEQ ID NO: 56 | 3′ | TCGC ACT |
| SEQ ID NO: 57 | 3′ | TCGC AC |
| SEQ ID NO: 58 | 3′ | TCGC A |
| SEQ ID NO: 59 | 3′ | TCGC |
| SEQ ID NO: 60 | 3′ | TCG |
| SEQ ID NO: 61 | 3′ | TC |
| SEQ ID NO: 62 | 3′ | T |

### FULL CITATIONS FOR DOCUMENTS REFERRED TO IN THE DISCLOSURE

[1] a) A. D. Ellington, J. W. Szostak, Nature 1990, 346, 818-822; b) C. Tuerk, L. Gold Science 1990, 249, 505-510; c) D. L. Robertson, G. F. Joyce, Nature 1990, 344, 467-468.
[2] a) D. S. Wilson, J. W. Szostak, Annu. Rev. Biochem. 1999, 68, 611-647; b) S. M. Shamah, J. M. Healy, S. T. Cload, Acc. Chem. Res. 2008, 41, 130-138; c) M. R. Gotrik, T. A. Feagin, A. T. Csordas, M. A. Nakamoto, H. Tom Soh, Acc. Chem. Res. 2016, 49, 1903-1910; d) H. Gu, K. Furukawa, Z. Weinberg, D. F. Berenson, R. R. Breaker, J. Am. Chem. Soc. 2013, 135, 9121-9129.
[3] a) J. Liu, Z. Cao, Y. Lu, Chem. Rev. 2009, 109, 1948-1998; b) W. Tan, M. J. Donovan, J. Jiang, Chem. Rev. 2013, 113, 2842-2862; c) M. Famulok, J. S. Hartig, G. Mayer, Chem. Rev. 2007, 107, 3715-3743.
[4] a) P. R. Bohjanen, R. A. Colvin, M. Puttaraju, M. D. Been, M. A. Garcia-Blanco, Nucleic Acids Res. 1996, 24, 3733-3738; b) D. A. Di Giusto, G. C. King, J. Biol. Chem. 2004, 279, 46483-46489; c) X. Tang, M. Su, L. Yu, C. Lv, J. Wang, Z. Li, Nucleic Acids Res. 2010, 38, 3848-3855; d) D. A. Di Giusto, S. M. Knox, Y. Lai, G. D. Tyrelle, M. T. Aung, G. C. King, ChemBioChem 2006, 7, 535-544; e) H. Kuai, Z. Zhao, L. Mo, H. Liu, X. Hu, T. Fu, X. Zhang, W. Tan, J. Am. Chem. Soc. 2017, 139, 9128-9131; f) H. Dong, L. Han, Z. Wu, T. Zhang, J. Xie, J. Ma, J. Wang, T. Li, Y. Gao, J. Shao, P. J. Sinko, L. Jia, Chem. Mater. 2017, 29, 10312-10325.
[5] a) A. Fire, S. Q. Xu, Proc. Natl. Acad. Sci. USA 1995, 92, 4641-4645; b) D. Liu, S. L. Daubendiek, M. A. Zillman, K. Ryan, E. T. Kool, J. Am. Chem. Soc. 1996, 118, 1587-1594; c) W. Zhao, M. M. Ali, M. A. Brook, Y. Li, Angew. Chem. Int. Ed. 2008, 47, 6330-6337; Angew. Chem. 2008, 120, 6428-6436; d) M. Liu, W. Zhang, Q. Zhang, J. D. Brennan, Y. Li, Angew. Chem. Int. Ed. 2015, 54, 9637-9641; Angew. Chem.2015, 127, 9773-9777; e) M. Liu, Q. Zhang, Z. Li, J. Gu, J. D. Brennan, Y. Li, Nat. Commun. 2016, 7, 12704; f) M. Liu, C. Y. Hui, Q. Zhang, J. Gu, B. Kannan, S. Jahanshahi-Anbuhi, C. D. M. Filipe, J. D. Brennan, Y. Li, Angew. Chem. Int. Ed. 2016, 55, 2709-2713; Angew. Chem. 2016, 128, 2759-2763; g) M. Liu, Q. Zhang, D. Chang, J. Gu, J. D. Brennan, Y. Li, Angew. Chem. Int. Ed. 2017, 56, 6142-6146; Angew. Chem. 2017, 22, 6238-6242.
[6] a) D. A. Di Giusto, W. A. Wlassoff, J. J. Gooding, B. A. Messerle, G. C. King, Nucleic Acids Res. 2005, 33, e64; b) L. Wang, K. Tram, M. M. Ali, B. J. Salena, J. Li, Y. Li, Chem. Eur. J. 2014, 20, 2420-2424.
[7] a) B. Stoddart, M. H. Wilcox, Curr. Opin. Infect. Dis. 2002, 15, 513-518; b) N. Shetty, M. W. D. Wren, P. G. Coen, J. Hosp. Infect. 2011, 77, 1-6; c) S. D. Goldenberg, M. Gumban, A. Hall, A. Patel, G. L. French, Diagn. Microbiol. Infect. Dis. 2011, 70, 417-419.
[8] a) T. D. Wilkins, D. M. Lyerly, J. Clin. Microbiol. 2003, 41, 531-534; b) L. Fenner, A. F. Widmer, G. Goy, S. Rudin, R. Frei, J. Clin. Microbiol. 2008, 46, 328-330; c) J. R. Ticehurst, D. Z. Aird, L. M. Dam, A. P. Borek, J. T. Hargrove, K. C. Carroll, J. Clin. Microbiol. 2006, 44, 1145-1149; d) F. C. Tenover, E. J. Baron, L. R. Peterson, D. H. Persing, J. Mol. Diagn 2011, 13, 573-582.
[9] a) M. Liu, Q. Yin, J. D. Brennan, Y. Li, Biochimie 2018, 145, 151-157; b) C. Y. Hui, M. Liu, Y. Li, J. D. Brennan, Angew. Chem. Int. Ed. 2018, 57, 4549-4553; Angew. Chem. 2018, 130, 4639-4643.
[10] Y. Li, C. R. Geyer, D. Sen, Biochemistry 1996, 35, 6911-6922.
[11] B. M. Anderson, C. D. Anderson, R. L. Vantassell, D. M. Lyerly, T. D. Wilkins, Arch. Biochem. Biophys. 1993, 300, 483-488.
[12] a) T. Planche, A. Aghaizu, R. Holliman, P. Riley, J. Poloniecki, A. Breathnach, S. Krishna, Lancet Infect. Dis. 2008, 8, 777-784; b) D. L. McCollum, J. M. Rodriguez, Clin. Gastroenterol. Hepatol.2012, 10, 581-592.
[13] M. Kawada, M. Annaka, H. Kato, S. Shibasaki, K. Hikosaka, H. Mizuno,Y. Masuda, T. Inamatsu, J. Infect. Chemother. 2011, 17, 807-811.
[14] M. Liu, Q. Zhang, Z. Li, J. Gu, J. D. Brennan, Y. Li, Nat. Commun. 2016, 7, 12074.

1. A method of identifying or producing an aptamer capable of binding to a target molecule, wherein the method comprises:
a) providing a plurality of circular nucleic acid molecules, wherein the plurality of circular nucleic acid molecules comprises oligonucleotides having at least one random nucleotide domain flanked by a 5′-end primer region and a 3′-end primer region;
b) contacting the plurality of circular nucleic acid molecules with a bare solid support;
c) collecting unbound circular nucleic acid molecules;
d) contacting the unbound circular nucleic acid molecules with solid support coated with the target molecule to form a complex comprising bound circular nucleic acid molecules;
e) optionally washing the complex;
f) eluting the bound circular nucleic acid molecules from the complex; and
g) amplifying the eluted circular nucleic acid molecules by polymerase chain reaction (PCR).

2. The method of claim 1, further comprising repeating steps d) to g) for one to nineteen times, optionally for six to eleven times, with the amplified eluted circular nucleic acid molecules of g) as the unbound circular nucleic acid molecules in step d).

3. The method of claim 1, wherein providing a plurality of circular nucleic acid molecules comprises preparing a plurality of circular nucleic acid molecules by circularizing a plurality of linear nucleic acid molecules.

4. The method of claim 1, wherein the solid support comprises nitrocellulose filter disc, optionally with a pore size 0.45 or 0.22 um, or magnetic beads coated with a metal, optionally Nickel Nitrilotriacetic acid magnetic beads (NiMBs).

5. The method of claim 1, wherein step f) eluting comprises denaturing the circular nucleic acid molecules, optionally wherein the denaturing comprises heating or urea treatment, optionally 10M urea.

6. The method of claim 1, wherein the target molecule is a microorganism target, or a target molecule present on or generated from a microorganism or a virus.

7. The method of claim 6, wherein the microorganism is selected from the group consisting of a bacteria, fungi, archaea, protists, algae, plankton and planarian.

8. The method of claim 6, wherein the microorganism is a pathogenic bacterium.

9. The method of claim 8, wherein the pathogenic bacterium is selected from the group consisting of Escherichia coli O157:H7, Listeria monocytogenes, Salmonella typhimurium or Clostridium difficile.

10. The method of claim 1, wherein the target molecule is selected from the group consisting of small inorganic molecule, small organic molecule, metal ion, biomolecule, toxin, biopolymer, optionally nucleic acid, carbohydrate, lipid, peptide, protein, optionally glutamate dehydrogenase.

11. The method of claim 1, wherein the oligonucleotides include a primer region to allow for amplification and a random single stranded DNA sequence domain of about 20 to about 80 nucleotides, optionally about 40 nucleotides, and wherein the primer region comprises sequences of SEQ ID NOs: 2 or 63, and 64.

12. A circular DNA aptamer that binds to C. difficile glutamate dehydrogenase (GDH), wherein the circular DNA aptamer comprises a sequence of SEQ ID NO: 6, 7, 14, or 16, or a functional fragment or modified derivative thereof.

13. An aptamer probe comprising the circular DNA aptamer of claim 12 and a detectable label, optionally the detectable label is a fluorescent moiety, optionally the fluorescent moiety is a fluorophore.

14. A biosensor system comprising:
the circular DNA aptamer of claim 12 attached directly or indirectly to a solid support.

15. The biosensor system of claim 14, further comprising components for detecting a signal through rolling circle amplification (RCA) of the circular DNA aptamer in a test sample.

16. A method for detecting the presence of a target C. difficile glutamate dehydrogenase (GDH) in a test sample, comprising:
a) contacting the test sample with a circular DNA aptamer of claim 12 to form a mixture, wherein the circular DNA aptamer is capable of binding to the target C. difficile glutamate dehydrogenase to form a complex;
b) separating the complex from the mixture; and
c) detecting a signal from the complex through rolling circle amplification (RCA) of the circular DNA aptamer,
wherein detection of a signal indicates the presence of the target molecule in the test sample and the lack of signal indicates the absence of the target molecule.

17. A method of detecting C. difficile infection in a subject comprising:
a) testing a sample from the subject for the presence of C. difficile GDH by the method of claim 16; and
b) if GDH is present, further comprising testing the sample for the presence of C difficile toxins A and B,
wherein the presence of GDH and the presence of toxins A and B indicate that the subject has a C. difficile infection.

18. The method of claim 17, wherein the testing for the presence of C. difficile toxins A and B is selected from the group consisting of cell cytotoxicity neutralization assay, toxin enzyme immunoassay or detection of toxin genes using PCR.

19. The method of claim 17, further comprising treating the subject for C. difficile infection if GDH and toxins are present.

20. A kit for detecting C. difficile GDH, wherein the kit comprises the circular DNA aptamer of claim 12 and instructions for use of the kit.
